(19) United States
(12) Patent Application Publication
(10) Pub. No.: US 2005/0090829 A1
(43) Pub. Date: Apr. 28, 2005

(54) Spinal bone chisels

(75) Inventors: Erik Martz, Savage, MN (US); Joel Millets, Eatontown, NJ (US)

Correspondence Address: William Squire, Esq.;c/o Carella, Byme, Bain, Gilfillan, Cecchi,, Stewart & Olstein, 5 Becker Farm Road, Roseland, NJ, 07068, US

(73) Assignee: Osteotech, Inc.

(21) Appl. No.: 10/808,186

(22) Filed: Mar. 23, 2004

Related U.S. Application Data
(60) Provisional application No. 60/513,893, filed on Oct. 23, 2003.

(51) Int. Cl. A61B 17/32; A61B 17/00

(52) U.S. Cl. 606/79; 606/167

(57) ABSTRACT

A chisel with distal cutting edges, used to form channels in adjacent vertebrae, includes a shank having a longitudinal axis and a cutting head coupled to the distal end of the shank. The cutting head includes distal cutting edges, which may be linear, arcuate, or V-shaped, and a guide member, which may be hollow or formed as two spaced members, to guide the chisel into the disc space to uniformly chisel both adjacent vertebrae simultaneously to partially form a channel in the vertebrae. The guide member and cutting head may have openings for distributing cut debris to the top and bottom surfaces of the head. The guide member may also include distal transverse, longitudinal side and/or vertical cutting edges, some of which may be in stepped relationship, for further cutting the endplates and removing disc material from between adjacent vertebrae, and for scraping of endplates. Other embodiments are disclosed.

This application claims the benefit of provisional application Ser. No. 60/513,893 filed Oct. 23, 2003, incorporated by reference herein in its entirety.

This invention relates to spinal implant chisel tools and method of preparing the spinal disc space, and more particularly to spinal chisel instruments having various cutting edges for preparing the intervertebral disc space for implants.

### CROSS REFERENCE TO RELATED APPLICATIONS AND PATENTS

Of interest are commonly owned copending provisional application Ser. No. 60/397,232 filed Jul. 19, 2002 in the name of David Chow et al., and PCT application Serial No. PCT/US03/21967 filed Jul. 11, 2003, entitled Spinal Implant Insertion Adjustment Instrument and Implants for use therewith, in the name of Osteotech Inc., all incorporated by reference herein.

During spinal surgery, the surgeon may approach the spine from a variety of different orientations. One orientation uses the posterior approach, another uses the anterior approach and others may approach laterally, posterior or anterior or antero-lateral, an angle somewhere between the anterior and lateral approaches.

U.S. Pat. No. 6,610,065 to Branch et al. discloses instruments for performing surgical procedures relating to lumbar interbody fusion. A box chisel is provided for preparation of the preformed cavity in the intervertebral disc space. The box chisel includes a handle, having an engagement hole adapted for attachment of an impacting tool such as a slap hammer. The box chisel also includes a shaft extending from the handle and connecting with a cutting head. The cutting head includes a first arm and opposing second arm extending from the shaft substantially parallel to the longitudinal axis. An upper cutting blade and opposing lower cutting blade are disposed between first and second arms which define an internal cavity for receipt of bone chips and cutting debris.

U.S. Pat. No. 4,697,586 to Gazale discloses a combined chisel-guide surgical instrument for performing osteotomy and other procedures on the human vertebra comprising at least one longitudinally directed and movable chisel each including at least one front cutting edge for penetrating into the vertebra. The instrument further includes a longitudinally directed guide including a front guide tip being locatable within the intervertebral space for accommodating and directing the motion of the chisel cutting edges into the vertebra.

European Patent Application No. EP 1,308,132A2 to Depuy AcroMed, Inc. (Rogers et al.) discloses a vertebral endplate chisel which may be used in a Posterior Lumbar Interbody Fusion procedure. In one embodiment, a box chisel device includes a base portion, an intermediate longitudinal portion, and a handle portion located proximal to the proximal portion of the base. Upper and lower portions terminate in distally-extending upper and lower shavers. The intermediate portion terminates distally in a flat thin guide having a head portion extending from a neck which extends from the intermediate portion. The head portion includes upper and lower land portions, upper and lower tapered portions, and a flat leading edge. The tips of the shavers terminate distally before the head portion forms its lands.

US Patent Application Publication No. 2002/0068941A1 to Hanson et al., discloses instruments and methods for preparing adjacent bones for fusion and for inserting implants. A distal end of the instrument includes a first cutting edge, a second cutting edge, and third and fourth cutting edges. The first and second cutting edges extend distally beyond the third and fourth cutting edge.

U.S. Pat. No. 6,096,038 to Michelson discloses distraction tools for distraction of adjacent vertebrae, implants for insertion into the spine, drills for drilling the intervertebral site to prepare the site for implant insertion, other tools used for preparing the disc space by cutting bone, a driver extraction instrument for extracting an implant driver tool from the spinal disc space and generally discloses surgery for providing an integrated discectomy, fusion and interbody internal spinal fixation.

U.S. Pat. No. 6,174,311 to Branch discloses implants formed from donor bone for use in lumbar interbody fusion procedures and instruments for performing such procedures. Specific implants and instruments are disclosed for inserting the implants and for preparing the intervertebral space to receive the implants. Disclosed is a box chisel that has a hollow core that is somewhat rectangular.

Also disclosed is a plane scraper and a rotatable cutter. This latter cutter has multiple cutting arms defining a cavity therebetween for receiving cutting debris. Each arm has at least two cutting blades. The blades extend axially between the handle and the cutting end. The box chisel cutting edges are normal to the axial direction of the tool in a direction from the handle to the box cutter, whereas the rotating cutter cutting edges are parallel to the axial direction. In use, this rotating cutter tool cuts bone by rotation of the tool about its longitudinal axis.

In another embodiment, a box chisel is disclosed that has a depth stop to prevent the chisel from cutting deeper into the disc space than a predetermined depth and includes depth indicator marks to indicate the depth of penetration of the chisel. Implants and implant holders are also disclosed. This patent is incorporated by reference in its entirety.

U.S. Pat. No. 4,697,586 to Ganzale discloses a combined chisel-guide surgical instrument. The instrument is for performing osteotomy and other procedures on the human vertebra and comprises at least one longitudinally directed and movable chisel each including at least one front cutting edge for penetrating into the vertebra, a longitudinally directed guide including a front guide tip being locatable within intervertebral space for accommodating and directing the motion of the chisel cutting edges into the vertebra, a handle fixedly secured to rear extension of the guide for directing and placing the guide tip into the intervertebral space, a front impact block member connected to the rear extension of the chisel, an intermediate longitudinally directed cylindrical member connected to the rear end of the front impact block member, a rear impact cylindrical member fixedly connected to the rear end of the intermediate cylindrical member, and a longitudinally movable impact hammer accommodated by the intermediate cylindrical member.

The impact hammer causes forward penetration of the chisel front cutting edge to the desired penetration depth and the impact hammer impacts the cylindrical member to cause rearward retraction of the chisel and the handle causes rearward retraction of the guide tip from the disc space. The guide tip serves as a depth gauge. Surface extensions at the rear of the guide tip prevent penetration deeper than the anterior longitudinal ligament.

The chisel slides along a track surface on the guide. A two chisel embodiment is disclosed wherein one chisel penetrates one vertebra or two chisels are used to penetrate two vertebra. The impact hammer is operative with the one chisel or two chisels which are arranged in mirror image fashion to each other and are each disclosed as U-shape in one embodiment.

The guide tip is inserted into the disc space first. The chisel is then slid onto the handle and along a surface of the guide until the cutting edges rest on the dorsal aspect of the vertebral space. The impact hammer is used to insert the cutting edges into the vertebral plates. The chisel is withdrawn with the hammer leaving the guide tip inserted in the disc space. The tip is then withdrawn. In a two chisel mode, the guide tip is inserted first and then either or both chisels may be operated at the same time wherein the chisels may be driven one at a time or together. The guide tip is removed after the chisels are removed. The cutting edges are normal to the insertion direction and longitudinal axis of the instrument similar to a box chisel.

U.S. Pat. No. 4,736,738 to Lipovsek et al. discloses an instrument kit and method for performing posterior lumbar interbody fusion. The kit includes first and second chisels and first and second shafts, a retaining ring with a set screw, an extraction hammer, a tamper and a hook. The first and second chisels each have a U-shaped blade and a shoulder between the blade and shaft. The second chisel is larger than the first chisel to enlarge the groove made by the first chisel. A stop prevents the shaft from slipping through the intervertebral space. The shoulders limit the depth of penetration of the chisels. The first chisel is used first and then withdrawn from the disc space. Then the second chisel is inserted to enlarge the channel formed by the first chisel. The chisel edges are coplanar and at right angles to the longitudinal axis of the instrument shaft.

U.S. Pat. No. 695,783 discloses a coping tool or chisel having a contour of molding to be cut and comprises a double chisel. A guide piece slides in a vertical recess in a frame of a mortising machine. A guide piece and gauge piece supported by the guide piece are fixed to the chisel. When a corner of the mold engages the guide piece the required depth is cut.

U.S. Pat. No. 740,937 discloses a chisel with a forward end with projecting spurs having rounded cutting edges. A forward end portion has a cutting edge.

U.S. Pat. No. 3,848,601 to Ma et al. discloses an interbody fusion apparatus including an intervertebral mortising chisel with an inner drill bit. The sides of the chisel have stops. The cutting edges are coplanar and lie in a plane normal to the longitudinal axis of the shaft forming what is generally referred to as a box chisel in that the edges of the chisel resemble a box shape.

U.S. Pat. No. 5,722,977 to Wilhelmy discloses a quadrilateral osteotome for use with a guide spacer. The guide spacer is inserted into the disc intervertebral space and while inserted, the chisel is then inserted to perform the bone cutting process guiding the chisel at this time. The guide spacer is received within the chisel hollow core and guides the chisel during its use. The chisel is shown as a box chisel.

U.S. Pat. No. 6,224,607 to Michelson discloses an instrument set that includes an extended guard for providing protected access to the disc space, and the adjacent surfaces of the adjacent vertebral bodies, a guide insertable into the guard, and a bone removal device such as a drill insertable into the guide.

A drawback of present cutting instruments is the lack of versatility in cutting spinal material from different adjacent areas.

A need is seen by the present inventors for a solution to this problem. A need is seen in particular for instrumentation that provides more versatility for chisel functions than present instrumentation. Another need is seen for a cutting instrument which can remove material from multiple adjacent areas during spinal surgery.

### SUMMARY OF THE INVENTION

According to an aspect of the present invention, a chisel is provided for preparing adjacent vertebrae for insertion of a spinal implant into the disc space defined by the vertebrae comprising a shank having a longitudinal axis and distal and proximal ends. A cutting head is coupled to the distal end of the shank, the cutting head including at least one wall extending substantially parallel to the longitudinal axis and at a first distance from the longitudinal axis. The at least one wall terminates distally the head with a first cutting edge lying in a plane for forming a channel in one of the vertebra. A cutting head is coupled to the distal end of the shank having a first wall terminating at a first cutting edge for cutting a channel in one of the vertebrae. A guide member extends from the head distally the cutting edge for insertion into the disc space and having a first leg portion extending substantially along the longitudinal axis and a second leg portion extending from the first portion in a direction transverse to the longitudinal axis defining a chamber therebetween for scooping cut material from between the vertebrae.

In a further aspect of the present invention, the first and second portions of the guide member integrally extend from a side wall of the cutting head and define a bend between the first and second portions.

In a further aspect, the cutting head includes a body defining an inner hollow portion. The body includes a side wall having an opening communicating with the inner hollow portion and communicating with a distal opening in the body to receive material scooped by the guide member.

In a further aspect, the cutting head includes a pair of opposing first cutting edges in spaced relation relative to the longitudinal axis and the guide member extends between the first cutting edges and traverses the longitudinal axis.

In a further aspect, the guide member has a distal end terminating with the at least one second cutting edge.

In a further aspect, the second cutting edge of the guide member further includes at least one side cutting edge extending along a periphery of the guide member and contiguous with the second cutting edge.

In a further aspect, the second cutting edge of the guide member is a second distance from the longitudinal axis which is less than the first distance from the longitudinal axis of the first cutting edges.

In another aspect, a chisel is provided for preparing adjacent vertebrae for insertion of a spinal implant into the disc space defined by the vertebrae comprising a shank having a longitudinal axis and distal and proximal ends, and a cutting head coupled to the distal end of the shank. The cutting head includes a body having at least one transversely cantilevered member having a first cutting edge, the cantilevered member extending substantially parallel to the longitudinal axis and being a first distance from the longitudinal axis. The cantilevered member terminates distally in the first cutting edge extending transversely to the longitudinal axis. The first cantilevered member and the head body defines an opening to provide visual access to a distal end of the cutting head. The first cutting edge of the first cantilever member lies in a plane for forming a channel in one of the vertebra. The cutting head further including a guide member positioned distal to the first cutting edge and extending along a plane substantially parallel to the longitudinal axis and positioned for bearing against adjacent vertebrae.

In a further aspect, the guide member includes at least one second cutting surface at a distal end terminating with a second cutting edge.

In a further aspect, the second cutting edge of the guide member further includes a cutting edge beveled away from the longitudinal axis and extending along a periphery of the guide member contiguously with the second cutting edge of the second cutting edge.

In a further aspect, the second cutting edge is a second distance from the longitudinal axis which is less then the first distance.

In a further aspect, the cutting head includes a pair of opposing first cutting edges in spaced relation about the longitudinal axis, a guide member extends between the cutting edges along a plane defined by a side wall of the cutting head such that an opening extends between the first cutting edges.

In a further aspect, the first cutting edge extends transversely to the longitudinal axis and is convex.

In a further aspect the first cutting edge extends transversely to the longitudinal axis and is concave.

In a further aspect the first cutting edge of the at least one cantilevered first member includes a second cutting edge extending at an angle from the first cutting edge in a direction transverse to the longitudinal axis.

In a further aspect the cutting head includes a pair of opposing cantilevered first members distally terminating in the first cutting edges in spaced relation about the longitudinal axis. The first cutting edges each include second cutting edges extending substantially perpendicular to the first cutting edges toward one another and in a direction transverse to the longitudinal axis.

In a further aspect the first cutting edge is curved in a direction transverse to the longitudinal axis.

In a further aspect the pair of opposing first surfaces are curved toward one another in a direction transverse to the longitudinal axis.

In another aspect, a chisel is provided for preparing adjacent vertebrae for insertion of a spinal implant into the disc space defined by the vertebrae where the chisel comprises a shank having a longitudinal axis and distal and proximal ends, and a cutting head coupled to the distal end of the shank. The cutting head includes at least one first side wall extending substantially parallel to the longitudinal axis and being a first distance from the longitudinal axis, the first side wall distally terminating with a first cutting edge lying in a plane for forming a channel in one of the vertebra. The cutting head further including a guide member positioned distal to the first member and at least one side extending substantially along a plane parallel to the longitudinal axis and having a distal wall traversing the longitudinal axis such that the guide member side and distal wall define a substantially central opening therebetween.

In a further aspect, the guide member includes a periphery surface around a perimeter of the substantially central opening, a second cutting edge extending along the periphery surface, the second cutting edge lying in a plane for removing material between adjacent vertebrae and juxtaposed distally beyond the first cutting edge, and the second cutting edge being a second distance from the longitudinal axis which is less than the first distance.

In a further aspect, the opening in the guide member is a channel partially defined by a web extending between at least two sides of the guide member, the cutting head includes an aperture proximally positioned in relation to the first cutting edge wherein the aperture communicates with the channel in the guide member.

In a further aspect, the at least one first cutting edge of the cutting head further includes a lower cutting edge in spaced opposing relation to an upper cutting edge with the longitudinal axis therebetween, the guide member further including a lower channel in opposing spaced relation to an upper channel, the cutting head further including a lower aperture in opposing spaced relation to an upper aperture and communicating with the lower channel.

In a further aspect, the cutting head further includes upper and lower first walls in opposing spaced relation about the longitudinal axis, and each of the upper and lower first walls terminating in a first cutting edge in mirror image relationship with each other.

In a further aspect, the cutting edge of a first wall is V-shaped and formed with two cutting edge legs distally interconnected at an apex of a V.

In a further aspect, the guide member defines a substantially central hollow opening extending therethrough and communicating with a substantially central opening in the guide member which extends therethough.

In another aspect of the present invention, a chisel is provided for preparing adjacent vertebrae for insertion of a spinal implant into the disc space defined by the vertebrae comprising a shank having a longitudinal axis and distal and proximal ends, and a cutting head coupled to the distal end of the shank. The cutting head including a first cutting portion comprising at least one first wall extending along a plane substantially parallel to the longitudinal axis and having a first distance from the longitudinal axis. The at least one first wall distally terminating with a first cutting edge lying in a plane for forming a channel in one of the vertebrae. The cutting head further including a guide member positioned distally to the first cutting edge and extending substantially along a substantially parallel plane to the longitudinal axis and having a side wall traversing the longitudinal axis. The guide member including a second cutting portion comprising at least one second cutting edge positioned for scraping material between vertebrae and juxtaposed distally beyond the first cutting edge, and a second distance between the at least one second cutting edges and the longitudinal axis being less then the first distance.

In a further aspect the guide member further includes a plurality of side walls defining the guide member and a second cutting portion, the second cutting portion of the guide member includes at least two cutting edges in spaced relation extending from the periphery of the side walls along substantially parallel planes with the longitudinal axis therebetween.

In a further aspect, the second cutting portion of the guide member includes two upper cutting edges in spaced relation extending from a periphery of the side walls along substantially parallel planes with the longitudinal axis therebetween, and the guide member includes two lower cutting edges in spaced relation extending from a periphery of the side wall as a mirror image of the two upper cutting edges.

In a further aspect, the guide member includes a web portion extending between the side walls of the guide member.

In a further aspect, the first cutting edge is serrated and is transverses the longitudinal axis.

In a further aspect the guide member defines a substantially central opening communicating with an opening positioned on a proximal side of the cutting head.

In another aspect of the present invention, a chisel instrument is provided for preparing adjacent vertebrae for insertion of a spinal implant into the disc space defined by the vertebrae, where the chisel comprises a shank having a longitudinal axis and distal and proximal ends, and a cutting head coupled to the distal end of the shank. The cutting head includes a first cutting portion comprising two side wall portions extending upwardly and in spaced relation to each other and a top wall connecting the two side wall portions such that an opening is defined between the top wall, side wall portions and the body of the cutting head. The side wall portions and top wall distally terminating in first cutting edges for forming a channel in at least one of the vertebrae. The cutting head further including a guide member positioned distal to the first cutting portion and extending substantially along the longitudinal axis and coextensive with the shank. The guide member having an end wall traversing the longitudinal axis.

In a further aspect, the guide member is defined by a plurality of side walls and includes a second cutting portion distal from a first cutting portion where the second cutting portion comprises at least one second cutting edge along the periphery of the wall of the guide member and the second cutting edge is a second distance from the longitudinal axis.

In a further aspect, the second cutting portion includes two upper cutting edges in spaced relation extending from the periphery of the side walls along substantially parallel planes with the longitudinal axis therebetween, and the guide member includes two lower cutting edges in spaced relation extending from a periphery of the side wall in mirror image relationship with the two upper cutting edges.

In a further aspect, at least one side wall of the guide member includes extends transversely to the longitudinal axis.

In a further aspect, the guide member includes a plurality of side walls defining a through channel extending substantially along the longitudinal axis and communicating with at least one opening in the cutting head in proximal relation to the first cutting edges, the side walls of the guide member distally terminating in second cutting edges.

In a further aspect, the body of the cutting head further includes an opening in a top wall thereof positioned adjacent to the distally terminating cutting edge of the top wall, the opening in the top surface of the cutting head communicating with the through channel in the head and a proximal opening in the head.

In a further aspect, the cutting head further includes an opening in a bottom wall of the cutting head in opposing spaced relation and communicating with an opening in a top wall of the cutting head such that the opening in the bottom wall is adjacent to a distally terminating cutting edge of the bottom wall in spaced mirror relationship with the opening in the top wall.

In another aspect of the present invention, a chisel is provided for preparing adjacent vertebrae for insertion of a spinal implant into the disc space defined by the vertebrae, the chisel comprising, a shank having a longitudinal axis and distal and proximal ends, and a cutting head coupled to the distal end of the shank. The cutting head including a first cutting portion comprising at least one first cutting edge extending substantially parallel to the longitudinal axis and being a first distance from the longitudinal axis. The first cutting edge distally terminating with a first cutting edge transverse to the longitudinal axis and lying in a plane for forming a channel in one of the vertebrae, the cutting head further including a guide member having a second cutting portion positioned distal to the first cutting portion and extending substantially along the longitudinal axis and terminating with a second cutting edge transverse to the longitudinal axis and lying in a plane for removing material between adjacent vertebrae and juxtaposed distally beyond the first cutting edge.

In a further aspect, the at least one first cutting edge includes multiple first cutting edges in stepped relation to one another.

In a further aspect, the cutting head includes a substantially central opening communicating with a distal end of the cutting head and communicating with an opening in a top surface of the cutting head proximal the at least one first cutting edges.

In a further aspect, the first cutting portion includes an upper cutting edge and a lower cutting edge in opposing space relation and a first distance from the longitudinal axis, and a second cutting portion includes an upper cutting edge and a lower cutting edge in opposing space relation and a second distance from the longitudinal axis which is less than the first distance, the cutting head defining a substantially central chamber which communicates with an opening in a distal end of the cutting head defined by the upper and lower cutting edges of the second cutting portion.

In a further aspect, the first cutting portion defines a first opening extending therethrough and communicating with the substantially central chamber in the cutting head.

In a further aspect, the second cutting portion includes a second cutting edge having two legs converging at a distal apex.

In a further aspect, the second cutting portion includes a second cutting side edge extending along a periphery of the second cutting portion and the cutting side edge facing in a direction substantially perpendicular to the longitudinal axis.

In a further aspect the second cutting portion includes upper and lower cutting edges in spaced opposite relation to one another and a side cutting edge extending contiguously along a periphery of the upper and lower cutting edges.

In a further aspect, the second cutting portion includes upper and lower cutting edges in spaced opposite relation to one another connected by a curved portion of the cutting head to form a U-shaped side surface with the curved portion at a proximal end and the distal ends of the legs of the U forming the second cutting edges, the U-shaped side surface having a cutting edge along at least a portion thereof.

In a further aspect, the first cutting edge includes two legs converging at a distal apex.

In a further aspect, the cutting head includes a top rasp surface.

In a further aspect, the cutting head includes a top surface having a plurality of raised sharpened projections.

In a further aspect, the cutting head includes a top surface having a plurality of raised cutting edges over a plurality of respective openings defined by the top surface such that material shaved by the cutting edges passes through the openings.

In a further aspect, the second cutting portion of the guide member includes a surface having a plurality of raised cutting edges over a plurality of respective openings in the second cutting portion such that the openings communicate with a substantially central opening communicating with the distal end of the head.

In a further aspect, the first cutting edge is curved toward the proximal end of the cutting head.

In a further aspect, the first cutting edge is angled such that an apex of the cutting edge faces toward the proximal end of the cutting head.

In another aspect of the present invention, a chisel instrument is provided for preparing adjacent vertebrae for insertion of a spinal implant into the disc space defined by the vertebrae. The chisel comprises a shank having a longitudinal axis and distal and proximal ends, a handle portion coupled to the proximal end of the shank, a cutting head coupled to the distal end of the shank. The cutting head including a hollow body being contiguous with an opening through the shank and an opening through the handle, the body of the cutting head comprising four sides distally terminating in cutting edges. A scalpel comprising a knob at a proximal end and a blade edge at a distal end. The scalpel being removable positioned through the contiguous openings of the handle portion, the shank, and the hollow body of the cutting head such that the blade edge extends distal to the cutting edges of the cutting head.

In a further aspect, the scalpel comprises a blade portion distally terminating in the blade edge, the blade portion coupled to a side of the distal end of the scalpel and extending along a plane substantially parallel to a longitudinal axis of the scalpel.

In a further aspect, the blade portion is planar and defines a width extending vertically.

In a further aspect, the blade portion is planar and defines a width extending horizontally.

In another aspect of the present invention, a chisel is provided for preparing adjacent vertebrae for insertion of a spinal implant into the disc space defined by the vertebrae, where the chisel comprises a shank having a longitudinal axis and distal and proximal ends, a handle portion coupled to the proximal end of the shank, and a cutting head coupled to the distal end of the shank, the cutting head including a hollow body comprising four sides distally terminating in quadrilateral cutting edges. A resilient member is positioned between a first proximal portion of the shank and a second distal portion of the shank.

In a further aspect, the resilient member is a spring.

In a further aspect, at least one supporting rod extending in a plane substantially parallel to the longitudinal axis is slidably connected to the shank, the supporting rod being fixedly connected to a proximal end of the cutting head.

In a further aspect an elongated guide element extending in a plane substantially parallel to the longitudinal axis is attached to an outer surface of the shank, a distal portion of the elongated guide element extending through the hollow body of the cutting head and distally terminating past the cutting edges and lying in a plane for insertion between adjacent vertebrae.

In a further aspect, the elongated guide element passes through an opening in the hollow body of the cutting head.

In a further aspect, the elongated guide element is planar and defines a width extending normal to the plane of the element.

In a further aspect, the elongated guide element is planar and defines a width extending vertically.

In another aspect of the present invention a chisel is provided for preparing adjacent vertebrae for insertion of a spinal implant into the disc space defined by the vertebrae, wherein the chisel comprises a shank having a longitudinal axis and distal and proximal ends, and a cutting head coupled to the distal end of the shank. The cutting head including upper and lower walls defining a first cutting portion distally terminating with first cutting edges, the first cutting edges being in spaced relation to each other with the longitudinal axis therebetween. The cutting head further includes a substantially central guide member positioned distal to the first cutting portion and extending substantially along the longitudinal axis and coextensive with the shank, the guide member having a side wall traversing the longitudinal axis, the guide member further including top and bottom surfaces in spaced relation to each other with the longitudinal axis therebetween and being a second distance from the longitudinal axis which is less than a first distance between the first cutting edges and the longitudinal axis. The cutting head further including upper and lower openings communicating with each other and extending through the cutting head, the upper and lower openings being defined by the upper and lower walls of the first cutting portion and the substantially central guide member.

In a further aspect, the chisel includes a plurality of supports extending between the upper wall and the guide member along a periphery of the cutting head defining an upper opening therebetween. The supports extending between the lower wall and the guide member along the periphery of the cutting head defining the lower opening therebetween.

In another aspect, the chisel of includes an upper support that extends between an upper wall and the guide member away from a periphery of the cutting head defining a plurality of upper openings with the periphery of the cutting head. A lower support extends between a lower wall and the guide member away from the periphery of the cutting head such that a plurality of lower openings are defined by the lower support and the periphery of the cutting head.

In another aspect of the present invention, a method is provided for preparation of a disc space for insertion of a spinal implant into the disc space between adjacent vertebrae, comprising the steps of positioning an extended guide member between adjacent vertebrae. The guide member being integral with a cutting head connected to a shank of a chisel instrument, the guide member directing first cutting edges of the cutting head into position with desired vertebrae. The method including removing a first portion of endplate material from at least one of two adjacent vertebrae using the first cutting edges of the cutting head. Removing a second portion of material including disc material between the adjacent vertebrae using at least one second cutting edge on a distal end of the guide member.

In a further aspect, the method including the step of scraping material from adjacent vertebrae using a third cutting edge extending along the periphery of the guide member by twisting the chisel about a longitudinal axis to turn the guide member in an arcuate path.

In a further aspect, the step of removing the second portion of material further includes the second portion of material being less distance to a longitudinal axis of the shank than the first portion of material such that the first cutting edges are positioned a first distance from the longitudinal axis to remove the first portion of material and the second cutting edges are positioned a second distance from the longitudinal axis which is less than the first distance and positions the at least one second cutting edge to remove the second portion of material.

### BRIEF DESCRIPTION OF THE DRAWINGS

FIG. 1 is a side elevation view of a chisel instrument according to an embodiment of the present invention including a shank having a handle and cutting head attached at opposite ends of the shank;

FIG. 2 is a cross sectional plan view taken along lines A-A of the instrument shown in FIG. 1 depicting the opening in the body of the cutting head;

FIG. 3 is an isometric view of the cutting head portion of the instrument shown in FIG. 1 depicting the openings in the body of the cutting head, proximate cutting edges, and the distal loading guide member;

FIG. 4 is an isometric view of another embodiment of a cutting head similar to the head shown in FIG. 3 where the guide member includes distal cutting edge;

FIG. 5 is a side elevation view of the cutting head shown in FIG. 4;

FIG. 6 is a cross sectional view of a guide member taken along line B-B in FIG. 4;

FIG. 7 is a plan view of another embodiment of a chisel instrument including a shank having a handle and cutting head attached at opposite ends of the shank;

FIG. 7a is an isometric view of another embodiment of a chisel instrument including a shank having a handle and cutting head attached at opposite ends of the shank;

FIG. 7b is a cross sectional plan view of the instrument of FIG. 7 taken along line a-a in FIG. 7c;

FIG. 7c is a side elevation view of the instrument shown in FIG. 7a;

FIG. 7d is a more detailed side elevation view of the cutting head of the instrument shown in FIG. 7c;

FIG. 8 is a side elevation view of the chisel instrument shown in FIG. 7;

FIG. 9 is a more detailed isometric view of the cutting head of the instrument shown in FIGS. 7 and 8 depicting the cantilever cutting edges and an opening therebetween, and a distal guide member;

FIG. 9a is a detail isometric view of another embodiment of a cutting head similar to the cutting head shown in FIG. 12, however, the cutting head includes additional distally facing cutting edges extending in a direction transverse to the longitudinal axis;

FIG. 10 is a more detailed plan view of the cutting head shown in FIG. 9;

FIG. 11 is a more detailed side view of the cutting head shown in FIG. 9;

FIG. 12 is an isometric view of another embodiment of a cutting head similar to the cutting head shown in FIG. 9 where the guide member includes cutting edges;

FIG. 13 is a plan view of the, cutting head shown in FIG. 12 depicting the cutting edges and the guide member;

FIG. 14 is a side elevation view of the cutting head shown in FIG. 12 and FIG. 14a shows an alternative embodiment;

FIG. 15 is a cross sectional front view of the guide member taken along line C-C in FIG. 14;

FIG. 16 is a plan view of another embodiment of a chisel instrument including a shank having a handle and cutting head attached at opposite ends of the shank;

FIG. 17 is a side elevation view of the chisel shown in FIG. 16;

FIG. 18 is a more detailed isometric view of the head of the chisel head of FIGS. 16-17 having convex cantilevered cutting members and a beveled distal guide member;

FIG. 19 is a plan view of the head shown in FIG. 18 depicting the convex cutting members;

FIG. 20 is a side elevation view of the head of FIG. 18;

FIG. 21 is an isometric view of another embodiment of a chisel head similar to the head of FIG. 18 where the beveled guide member includes cutting edges;

FIG. 21a is an alternative embodiment of the head of FIG. 21 having proximal convex cutting edges and guide member cutting edges;

FIG. 21b is a perspective view of an alternative embodiment of the cutting head of FIG. 21 having proximal curved cutting edges and guide member cutting edges;

FIG. 21c is a front elevation view of the cutting head of FIG. 21b;

FIG. 21d is a front elevation view of vertebrae cut by the cutting head of FIG. 21b;

FIG. 22 is a side elevation view of the head shown in FIG. 21 depicting the distal guide member cutting edges;

FIG. 23 is a front cross sectional view of the guide member taken along line D-D in FIG. 22;

FIG. 24 is a plan view of another embodiment of a chisel instrument including a shank having a handle and cutting head attached at opposite ends of the shank;

FIG. 25 is a side elevation view in cross section taken along line E-E of the chisel shown in FIG. 24;

FIG. 26 is a more detailed isometric view of the chisel head shown in FIG. 24 showing the guide member and proximal cutting edges of the head body;

FIG. 27 is a plan view of the head shown in FIG. 26 depicting the opening in the guide member;

FIG. 28 is a side view of the head in cross section taken along the line F-F shown in FIG. 27;

FIG. 29 is an isometric view of a chisel head similar to the head shown in FIG. 26 where the guide member includes cutting edges;

FIG. 30 is a plan view of the chisel head shown in FIG. 29 depicting the opening in the guide member;

FIG. 31 is a side elevation view in cross section taken along line G-G of the chisel head shown in FIG. 30 depicting upper and lower cutting edges;

FIG. 31a is a side elevation view in cross section of an alternative embodiment of the head shown in FIGS. 30 and 31 where the body of the head is hollow;

FIG. 32 is a plan view of another embodiment of a chisel instrument including a shank having a handle and cutting head attached at opposite ends of the shank;

FIG. 33 is a side elevation view in cross section taken along line H—H of the chisel instrument shown in FIG. 32;

FIG. 34 is a more detailed isometric view of the chisel head shown in FIG. 32 depicting the cutting edges and guide member and an opening in the body of the head;

FIG. 35 is a plan view of the chisel head shown in FIG. 34 depicting the openings in the body of the head including the guide member;

FIG. 36 is a side elevation view in cross section taken along line I-I of the head shown in FIG. 35;

FIG. 37 is a plan view of another embodiment of a chisel instrument including a shank having a handle and cutting head attached at opposite ends of the shank;

FIG. 38 is a side elevation view in cross section taken along line P-P of the chisel shown in FIG. 37;

FIG. 39 is a more detailed isometric view of the chisel head shown in FIGS. 37 and 38 depicting a triangular shaped cutting edge, an opening is the body of the chisel head, and a blunt tipped guide member having an opening;

FIG. 40 is a plan view of the chisel head shown in FIG. 39 showing the opening in the body of the head including the guide member;

FIG. 41 is a side elevation view in cross section taken along line Q-Q of the head shown in FIG. 40 showing the openings in the body of the chisel head including the guide member;

FIG. 42 is an isometric view of another embodiment of a chisel head similar to the head shown in FIG. 39 where the chisel head includes proximal V-shaped cutting edges and a guide member having distal longitudinal cutting edges;

FIG. 43 is a plan view of the chisel head shown in FIG. 42 showing the guide member distal cutting edges, the proximal cutting edges and the openings;

FIG. 44 is a side elevation view in cross section taken along the line R-R of the chisel head shown in FIG. 43 depicting the openings the body of the chisel head including the guide member;

FIG. 45 is a plan view of another embodiment of a chisel instrument including a shank having a handle and cutting head attached at opposite ends of the shank;

FIG. 45a is an isometric view of another embodiment of another embodiment of a chisel instrument including a shank having a handle and cutting head attached at opposite ends of the shank;

FIG. 45b is a side elevation view of the instrument of FIG. 45a;

FIG. 45c is a more detailed isometric view of the cutting head of FIGS. 45a and 45b;

FIG. 46 is a side elevation view in cross section along line J-J of the chisel shown in FIG. 45;

FIG. 47 is a more detailed isometric view of the chisel head shown in FIG. 45 showing the cutting edges and the guide member;

FIG. 48 is a plan view of the head shown in FIG. 47;

FIG. 49 is a side elevation view in cross section of the head along line K-K in FIG. 48;

FIG. 50 is an isometric view of another embodiment of a chisel where the body of the chisel head includes transverse cutting edges and a central guide member and spaced upper and lower distal cutting edges on the guide member;

FIG. 50a is an end elevation sectional view of the guide member of FIG. 50;

FIG. 51 is a plan view of the chisel head shown in FIG. 50;

FIG. 52 is a side elevation view in cross section taken along the line L-L of the chisel head shown in FIG. 51 showing the proximal and distal cutting edges;

FIG. 53 is a plan view of another embodiment of a chisel instrument including a shank having a handle and cutting head attached at opposite ends of the shank;

FIG. 54 is a side elevation view in cross section along line S-S of the chisel shown in FIG. 53;

FIG. 55 is a more detailed isometric view of the chisel head shown in FIGS. 53 and 54 depicting a saw toothed shaped proximal cutting edge and a guide member including longitudinally extending distal cutting edges;

FIG. 56 is a plan view of the chisel head shown in FIG. 55;

FIG. 57 is a side elevation view in cross section along line T-T of the chisel shown in FIG. 56;

FIG. 58 is a plan view of another embodiment of a chisel instrument including a shank having a handle and cutting head attached at opposite ends of the shank;

FIG. 59 is a side elevation view in cross section along line M-M of the chisel shown in FIG. 58;

FIG. 60 is a more detailed isometric view of the chisel head shown in FIG. 58 showing three upper and lower proximal cutting edges and a guide member;

FIG. 61 is a plan view of the chisel head shown in FIG. 60;

FIG. 62 is a side elevation view in cross section taken along line N-N of the head shown in FIG. 61;

FIG. 63 is an isometric view of another embodiment of a chisel head similar to the head shown in FIG. 60 where the guide member includes longitudinally extending distal cutting edges;

FIG. 64 is a plan view of the chisel head shown in FIG. 63;

FIG. 65 is a side elevation view in cross section taken along the line O-O of the chisel head shown in FIG. 64;

FIG. 66 is a plan view of another embodiment of a chisel instrument including a shank having a handle and cutting head attached at opposite ends of the shank;

FIG. 67 is a side elevation view in cross section along line U-U of the chisel shown in FIG. 66;

FIG. 68 is a more detailed isometric view of the chisel head shown in FIGS. 66 and 67 showing three proximal upper and lower cutting edges and a guide member including distal cutting sedges;

FIG. 69 is a plan view of the chisel head shown in FIG. 68 showing the opening in the body of the head;

FIG. 70 is a side elevation view in cross section taken along line V-V of the head shown in FIG. 69 depicting the openings in the body of the head, the guide member, and the cutting edges;

FIG. 71 is a plan view of another embodiment of a chisel instrument including a shank having a handle and cutting head attached at opposite ends of the shank;

FIG. 72 is a side elevation view in cross section along line W-W of the chisel shown in FIG. 71;

FIG. 73 is a more detailed isometric view of the chisel head shown in FIGS. 71 and 72 showing three upper and lower proximal cutting edges and a guide member including distal box cutting edges;

FIG. 74 is a plan view of the chisel head shown in FIG. 73 showing an opening in the body of the head;

FIG. 75 is a side elevation view in cross section taken along line X-X of the head shown in FIG. 74 showing the hollow inner portion of the head;

FIG. 76 is a plan view of another embodiment of a chisel instrument including a shank having a handle and cutting head attached at opposite ends of the shank including a longitudinal opening in the body of the head;

FIG. 76a is a side elevation view of another embodiment of a chisel instrument including a shank having a handle and a cutting head attached at opposite ends of the shank;

FIG. 76b is a plan view of the instrument shown in FIG. 76a;

FIG. 76c is a more detailed isometric view of the cutting head shown in FIG. 76a;

FIG. 76d is an isometric view of another embodiment of a chisel instrument including a shank having a handle and a cutting head attached at opposite ends of the shank;

FIG. 76e is a side elevation view of the chisel instrument shown in FIG. 76d;

FIG. 76f is a plan view in cross sectional taken along line P-P in FIG. 76e;

FIG. 76g is a more detailed isometric view of a portion of the cutting head shown in FIG. 76f;

FIG. 76h is a more detailed side elevation view of the cutting head shown in FIG. 76d;

FIG. 76i is a plan view of another embodiment of a cutting head having a curved cutting edge;

FIG. 76j is a detailed isometric view of a portion of the cutting head shown in FIG. 76i;

FIG. 76k is a plan view of another embodiment of a cutting head having a V-shaped cutting edge;

FIG. 76l is a detailed isometric view of a portion of the cutting head shown in FIG. 76k;

FIG. 76m is a plan view of another embodiment of a chisel instrument including a shank having a handle and cutting head attached at opposite ends of the shank where the cutting head includes a surface having a plurality of raised cutting edges and an opening in the body of the cutting head;

FIG. 76n is a detail isometric view of a portion of the cutting head shown in FIG. 76m;

FIG. 76o is a detail cross sectional view of the raised cutting edges taken along line M-M in FIG. 76n;

FIG. 76p is a detail cross sectional view of the raised cutting edges taken along line BB-BB in FIG. 76q;

FIG. 76q is a detail view of the raised cutting edges shown in FIG. 76n;

FIG. 77 is a side elevation view of the chisel shown in FIG. 76 showing distal and proximal cutting edges, the latter being in stepped relation;

FIG. 78 is a more detailed isometric view of the chisel head shown in FIGS. 76 and 77 showing the proximal stepped cutting edges and spaced guide members including distal cutting edges;

FIG. 79 is a side elevation view of the head shown in FIG. 78 showing the cutting edges;

FIG. 80 is a plan view of another embodiment of a chisel instrument including a shank having a handle and cutting head attached at opposite ends of the shank including a triangular distal cutting edge and an opening in the body of the cutting head;

FIG. 81 is a side elevation view of the chisel shown in FIG. 80;

FIG. 82 is a more detailed isometric view of the chisel head shown in FIGS. 80 and 81 showing proximal cutting edges and spaced guide members including distal arcuate cutting edges;

FIG. 83 is a side elevation view of the head shown in FIG. 78;

FIG. 84 is a plan view of another embodiment of a chisel instrument including a shank having a handle and cutting head attached at opposite ends of the shank;

FIG. 85 is a side elevation view of the chisel shown in FIG. 84;

FIG. 86 is a more detailed isometric view of the chisel head shown in FIGS. 84 and 85 showing transverse linear and V-shaped proximal cutting edges and distal guide members having arcuate distal cutting edges;

FIG. 87 is a side elevation view of the head shown in FIG. 86;

FIG. 88 is a plan view of another embodiment of a chisel instrument including a shank having a handle and cutting head attached at opposite ends of the shank;

FIG. 89 is a side elevation view of the chisel shown in FIG. 88;

FIG. 90 is a more detailed isometric view of the chisel head shown in FIGS. 88 and 89 showing multiple proximal transverse linear cutting edges and spaced guide members having arcuate distal cutting edges;

FIG. 91 is a side elevation view of the cutting head shown in FIG. 90;

FIG. 92 is a plan view of another embodiment of a chisel instrument including a shank having a handle and cutting head attached at opposite ends of the shank including a longitudinal opening in the body of the cutting head;

FIG. 93 is a side elevation view of the chisel shown in FIG. 92 showing the cutting edges;

FIG. 94 is a more detailed isometric view of the chisel head shown in FIGS. 92 and 93 including a body having a through longitudinal opening, a proximal cutting edge and a distal cutting edge on each of a pair of spaced cantilevered members;

FIG. 95 is a side elevation view of the head shown in FIG. 94;

FIG. 96 is a plan view of another embodiment of a chisel instrument including a shank having a handle and cutting head attached at opposite ends of the shank;

FIG. 97 is a side elevation view of the chisel shown in FIG. 92 including opposite spaced U-shaped side cutting edges;

FIG. 98 is a more detailed isometric view of the chisel head shown in FIGS. 92 and 93 including a body having a longitudinal through opening, upper and lower transverse proximal cutting edges, upper and lower distal cutting edges, and U-shaped opposite side cutting edges orthogonal to the proximal and distal edges;

FIG. 99 is an end elevation view of the head shown in FIG. 98;

FIG. 100 is a side elevation view of the head shown in FIG. 98 depicting the U-shaped side cutting edges;

FIG. 101 is a plan view of another embodiment of a chisel instrument including a shank having a handle and cutting head attached at opposite ends of the shank including a longitudinal opening in the cutting head;

FIG. 102 is a side elevation view of the chisel shown in FIG. 92;

FIG. 103 is a more detailed isometric view of the chisel head shown in FIGS. 101 and 102 including a body having a through opening, upper and lower proximal linear transverse cutting edges on cantilevered upper and lower members, and upper and lower distal chisel guides extending from the members;

FIG. 104 is a side elevation view of the head shown in FIG. 103;

FIG. 104a is a side elevation view of a cutting head having three cutting edges terminating with distal cutting edges;

FIG. 105 is a side elevation view of an embodiment of a box chisel instrument including a shank and a handle and cutting head at opposite ends of the shank;

FIG. 106 is a cross sectional plan view of the instrument shown in FIG. 105 taken along line Y-Y showing the box-shaped cutting head;

FIG. 107 is a front view of the instrument shown in FIGS. 105, 106 showing the center opening therethrough;

FIG. 108 is a side elevation view of a scalpel with blade for use with the box chisel shown in FIGS. 105-106;

FIG. 109 is a plan view of the scalpel shown in FIG. 108;

FIG. 110 is a front view of the scalpel and blade shown in FIG. 109;

FIG. 111 is an exploded view of the box chisel shown in FIGS. 105-106 with the scalpel shown in FIGS. 108-109;

FIG. 112 is a side elevation view of the combined box chisel and scalpel shown in FIG. 111;

FIG. 113 is a plan view of the combined box chisel and scalpel shown in FIG. 112;

FIG. 114 is a side elevation view of a box chisel instrument including a shank having a spring, and a handle and box-shaped cutting head attached at opposite ends of the shank;

FIG. 115 is a plan view of the box chisel instrument shown in FIG. 14 depicting the spring and a supporting rod;

FIG. 116 is an isometric view of the box chisel shown in FIGS. 114-115;

FIG. 117 is an isometric view of a box chisel instrument including a shank and a handle and box-shaped cutting head at opposite ends of the shank of the shank, where the cutting head includes an insertable guide;

FIG. 118 is a side elevation view of the insertable guide shown in FIG. 117;

FIG. 119 is a front view of the insertable guide shown in FIG. 118;

FIG. 120 is a plan view of the box chisel shown in FIG. 117 depicting a slot for insertion of the guide;

FIG. 121 is a side elevation view of the box chisel shown in FIG. 120 depicting a pin for attaching the insertable guide;

FIG. 122 is a front view of the box chisel shown in FIG. 120 depicting the slot;

FIG. 123 is a rear detail isometric view of the box chisel portion of the instrument shown in FIG. 120 depicting the slot;

FIG. 124 is a side view of an alternative chisel instrument including a shank having a handle and cutting head attached at opposite ends of the shank;

FIG. 125 is a more detailed elevation cross section view taken along line δ-δ in FIG. 124;

FIG. 126 is a front elevation view of the cutting head of the instrument shown in FIG. 124;

FIG. 127 is an isometric view of a cutting head of an alternative embodiment of the present invention;

FIG. 128 is an elevation cross sectional view of the embodiment of the cutting head of FIG. 127; and

FIG. 129 is a front elevation view of the cutting head shown in FIGS. 127 and 128.

### DETAILED DESCRIPTION OF THE INVENTION

In FIG. 1, chisel instrument 10 comprises an elongated shank 12 defining a longitudinal axis 14, a handle 16 attached to the shank 12 at a proximal end 17, and a cutting head 20 attached to the shank at a distal end 18. The handle 16 (shown in FIGS. 1 and 2) may have a bore 22 in which the shank 12 is inserted and attached to the handle. The shank 12 may include a stud 24 for receiving the handle. The shank 12 may be press fit into the handle bore 22 or may be threaded to the handle at bore 22 or may be bonded to the handle such as by welding. The handle 16 is elongated and extends along the longitudinal axis 14. The handle 16 has a transverse dimension greater than that of the shank 12 to permit ease of gripping by a surgeon during use. The handle and shank may be formed of stainless steel, for example, and preferably is circular cylindrical or, in the alternative may have other cross section shapes such as square or rectangular, for example. The handle 16 may also have flattened surfaces for receiving hammer blows used to manipulate the instrument 10 for manipulating an implant inserted into the intervertebral disc space.

As shown in FIG. 2, the cutting head 20 includes a guide member 25 having a first portion 25′ extending from a side wall of the cutting head and in a direction along longitudinal axis. The guide member further includes a distal curved portion 26 integral with the first portion 25′ and which extends transversely with respect to the longitudinal axis.

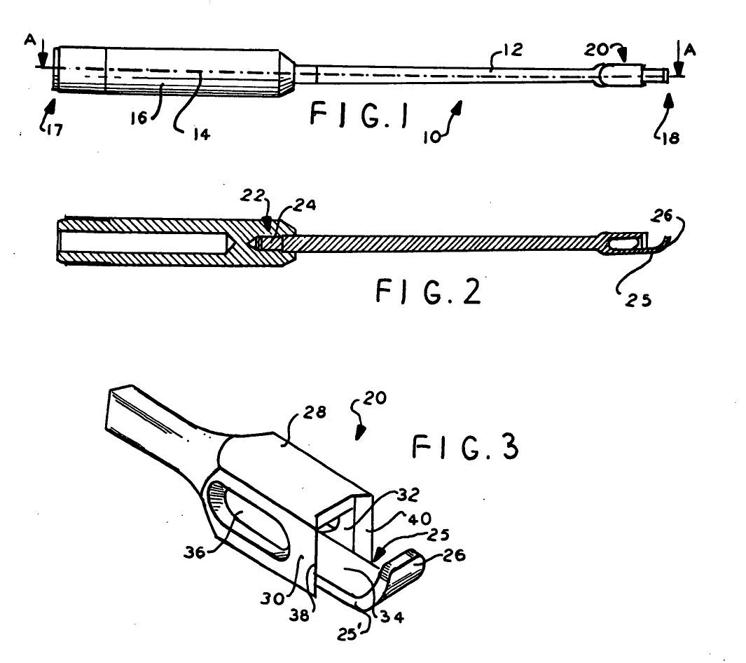

In FIG. 3, the cutting head 20 includes a side wall 28, top and bottom walls 30, 32, and a second side wall 34 from which extends distally the guide member 25 via portion 25′. The top and bottom walls 30, 32 each include openings 36 providing egress for material cut away during surgery. Cutting edges 38, 40, respectively, are at distal ends of the walls 30, 32. When the instrument is positioned as illustrated in FIG. 1, the guide member 25 extends between adjacent vertebrae uniformly guiding the cutting edges into the vertebrae. The cutting edges 38, 40 cut adjacent vertebrae while the curved portion of the guide member can scoop away the cut vertebrae and/or disc material.

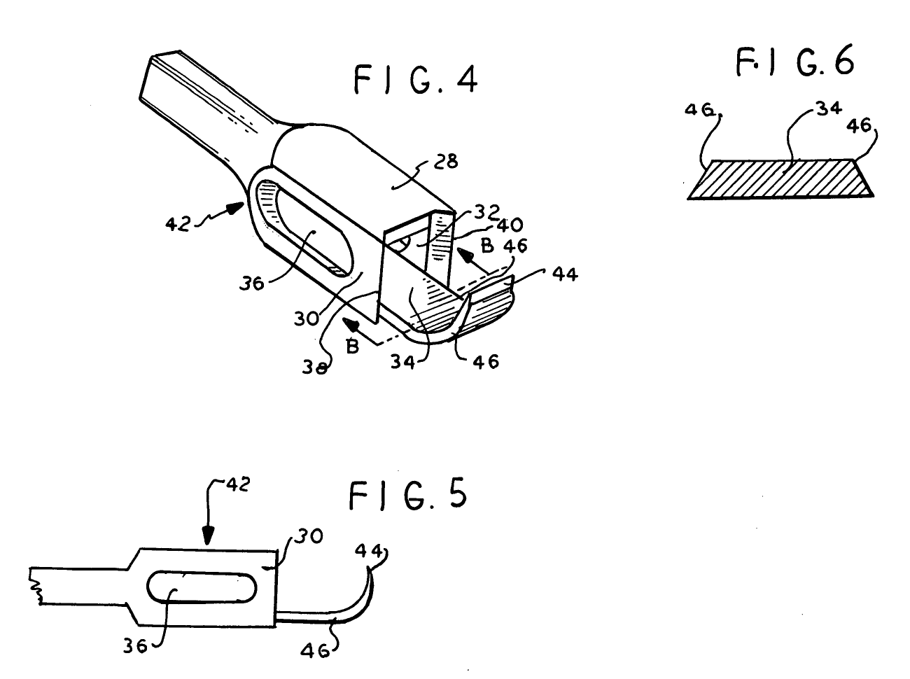

In FIG. 4, an alternative embodiment is shown. Cutting head 42 includes the same top and bottom surfaces and side walls as the cutting head 20 shown in FIG. 3, however, the guide member 34 terminates at an end cutting edge 44 and includes a cutting edge 46 along each of its sides. The cutting edges, shown in FIGS. 4-6, enable the guide member to be used during surgery for cutting, as well as, guiding the cutting edges 38, 40. The cutting edge 44 and side cutting edges 46 are positioned between the cutting edges 38, 40 of the top and bottom surfaces 30, 32 to cut away disc material between adjacent vertebrae. The side cutting edges 46 are closer to the longitudinal axis than the proximal cutting edges 38, 40. The side cutting edges may also be used to scrape away disc material and vertebrae material when the instrument is twisted about its longitudinal axis.

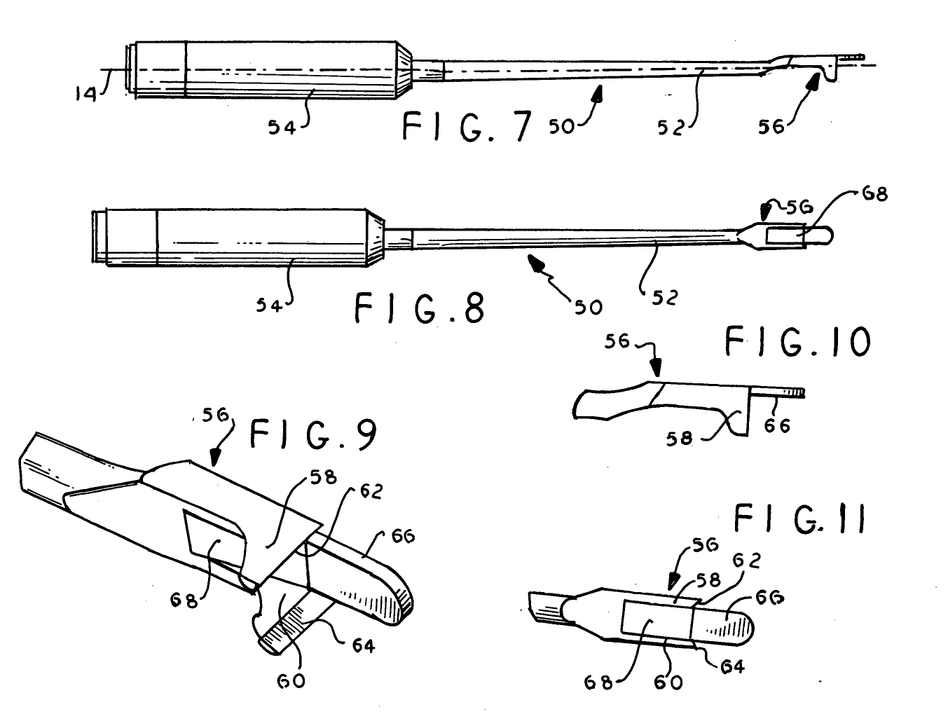

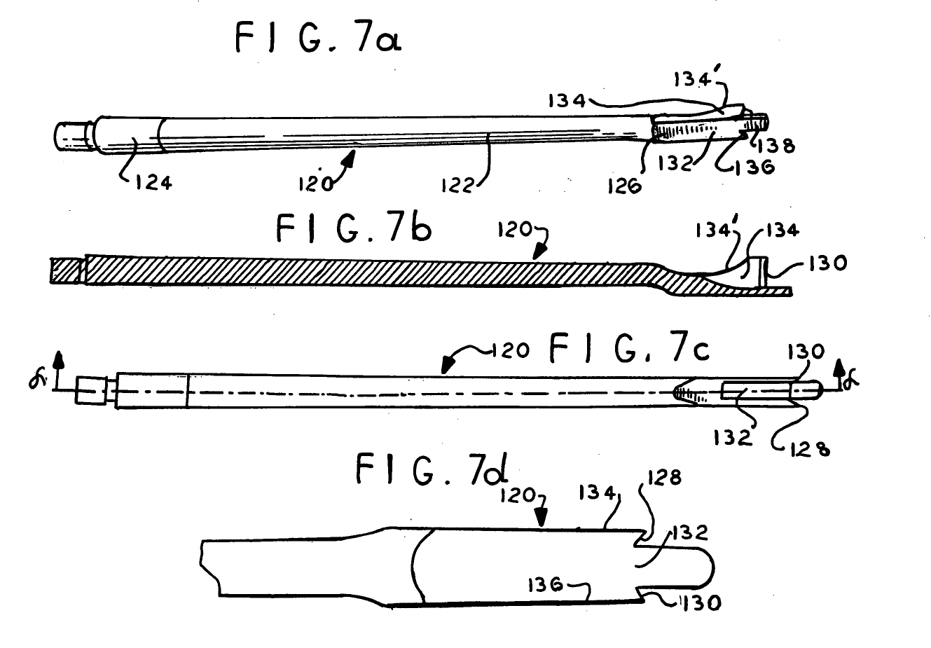

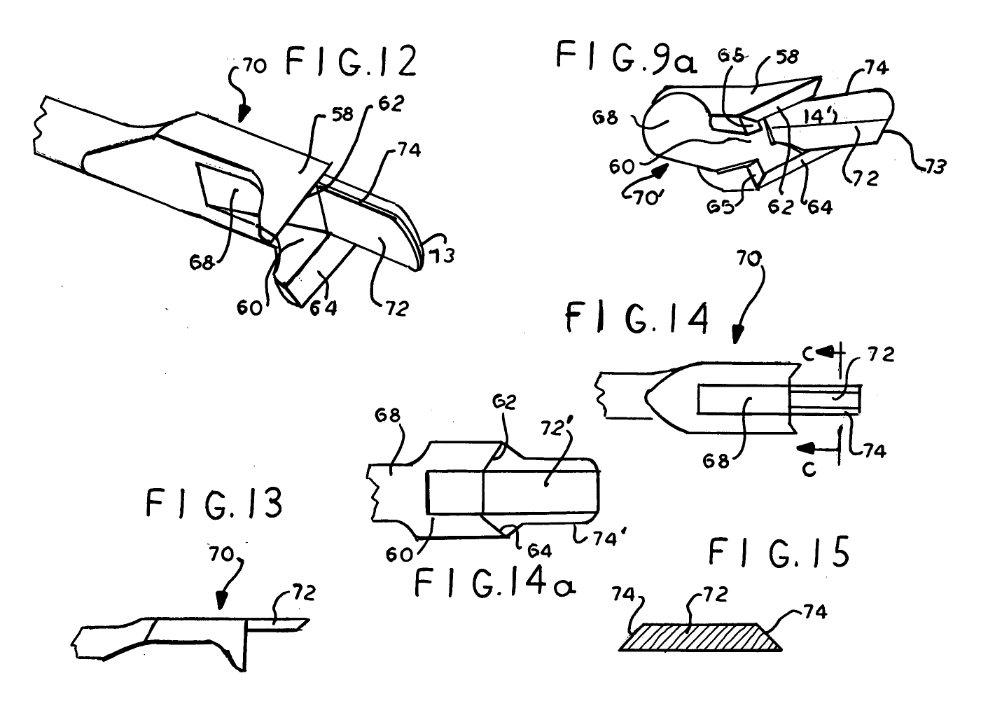

In another embodiment, an instrument 50 according to the present invention is shown in FIGS. 7-11. The shank 52 includes a handle 54 attached at a proximal end, and a cutting head 56 attached at a distal end as in the previous embodiment. The cutting head 56 in the embodiment shown in FIGS. 9-11, includes two transversely cantilevered members 58, 60 terminating distally with transverse linear parallel spaced cutting edges 62, 64 which are on opposite sides of the longitudinal shank axis. A guide member 66 extends from a side wall 68 between the cutting edges 62, 64. The transverse members 58, 60 extend from the head 56 side wall 68 and are free at their opposite ends spaced from the side wall 68. The opening between the members 58, 60 and side wall 68 is in the form of a channel and provides an open line of vision for a surgeon to observe the spinal area which is being worked on by the edges 62, 64. As can be seen in FIG. 9, the cutting edges 62, 64 are beveled away from the longitudinal axis equal distant to these edges. The angle of the cutting edge bevels may be set to a value to suit specified cutting needs.

Another embodiment of a cutting head is shown in FIG. 9a. The cutting head 70′ is similar to the head 156 shown in FIG. 9, except the cutting head 70′ includes distally facing opposed cutting edges 65 at the ends of the transverse members 58, 60 and the cutting edges 62, 64. The cutting edges 65 extend in a direction transverse to the direction of the longitudinal axis 14 shown in FIG. 7. They extend toward one another transverse to the edges 62, 64 forming corner edges and transverse to the edges 62, 64. The cutting edges 65 facilitate the cutting of material surrounding adjacent vertebrae. The cutting edges 65 are preferably perpendicular to the cutting edges 62, 64, as shown in FIG. 9a, however, the cutting edges 65 may also be at various other angles in relation to the cutting edges 62, 64.

Another embodiment of a cutting head 70 according to the present invention is shown in FIG. 12 and includes proximal cutting edges 62, 64 in cantilevered relation as shown in FIG. 9. However, planar guide member 72 terminates in an arcuate distal cutting edge 73. Also, the guide member includes a side cutting edge 74 which extends parallel to the shank longitudinal axis. The guide member cutting edges 73, 74, FIGS. 12-15, enables the guide member to be used during surgery for cutting as well as guiding the proximal cutting edges 62, 64. The guide member cutting edge 73 and side cutting edges 74 are positioned between the cutting edges 62, 64 of the cutting edges 58, 60 to cut away disc material between adjacent vertebrae. The side cutting edges 74 are closer to the central shank longitudinal axis 14′ than the cutting edges 62, 64 of the cutting edges 58, 60. The side cutting edge 74 may also be used to scrape away disc material and vertebrae material when the instrument is twisted about its longitudinal axis. The side cutting edges 73, 74 are beveled away from the longitudinal axis in a preferred embodiment, however, the angle of the cutting edge may be varied to suit the cutting need or beveled toward the longitudinal axis. FIG. 14a shows the guide member 72′ tapering toward the transverse upper and lower respective edges 62, 64 in an alternative embodiment.

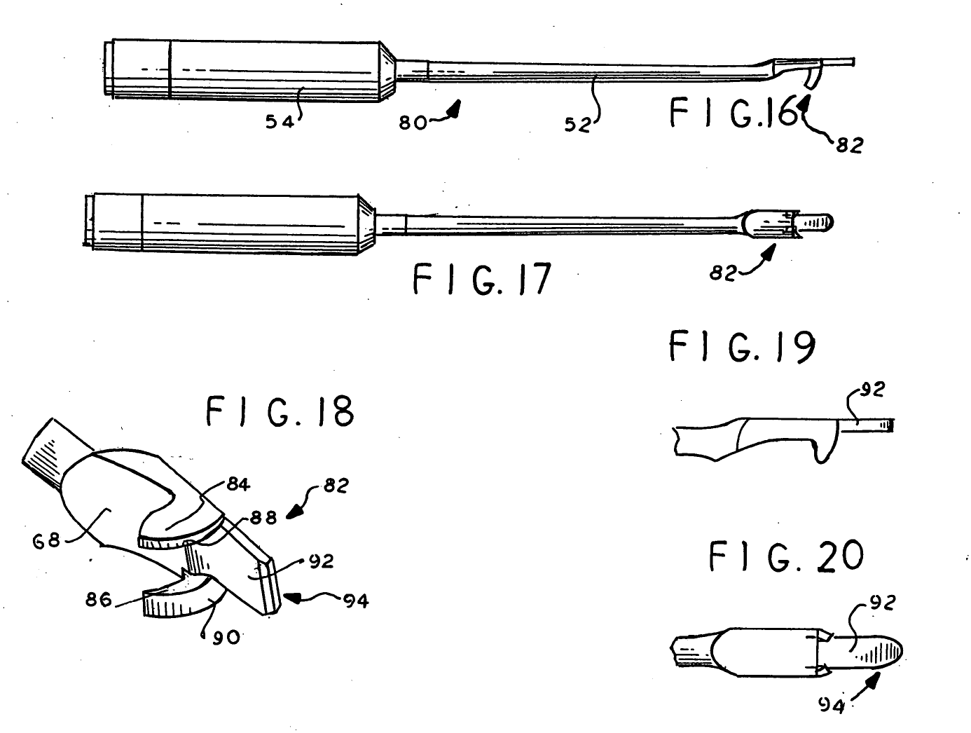

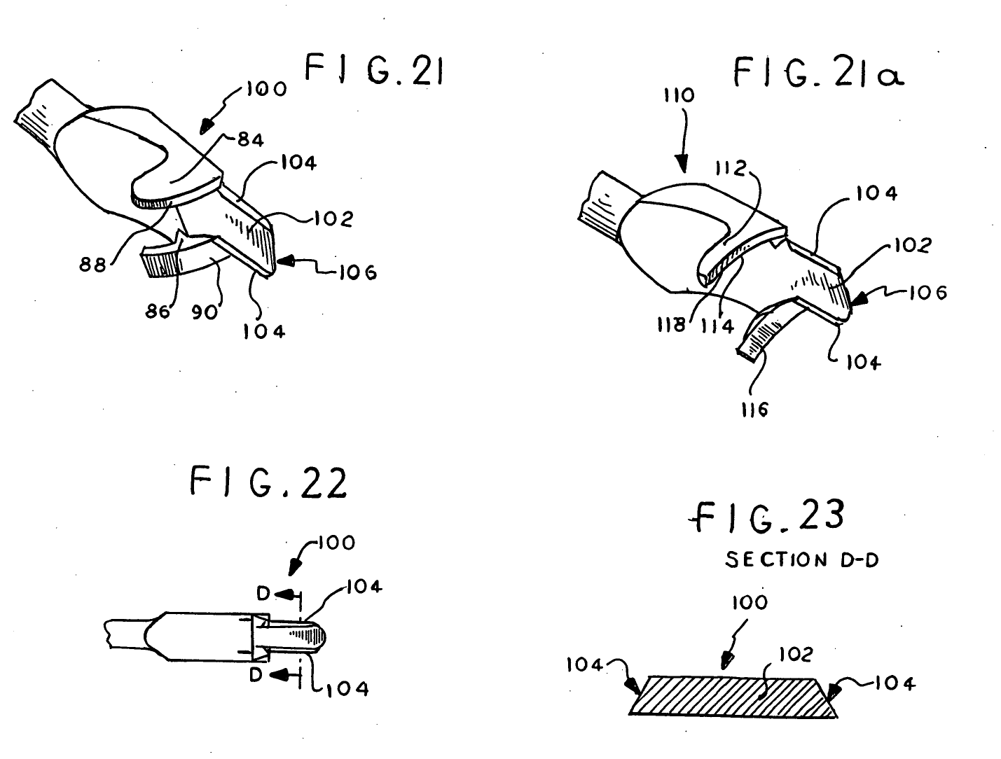

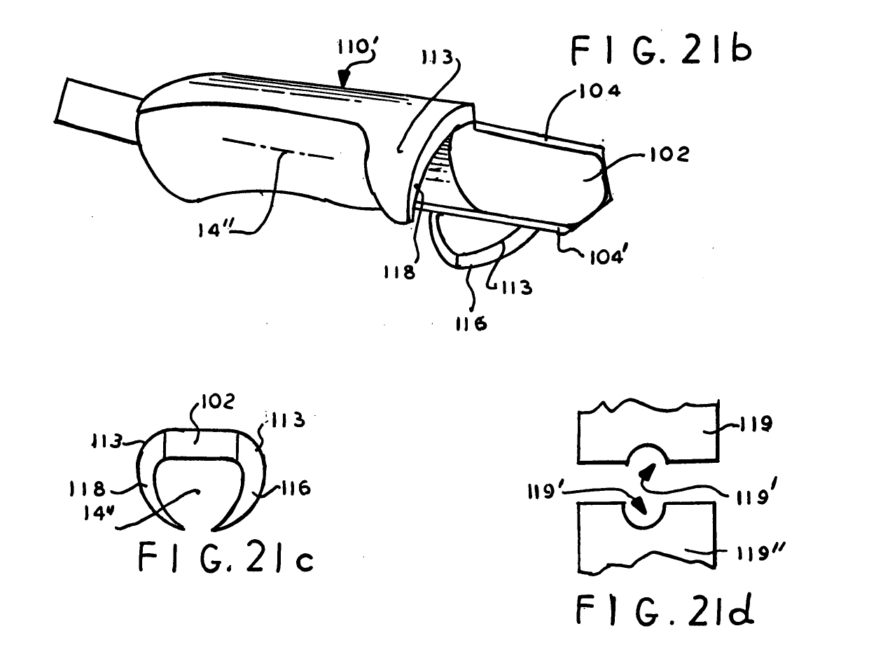

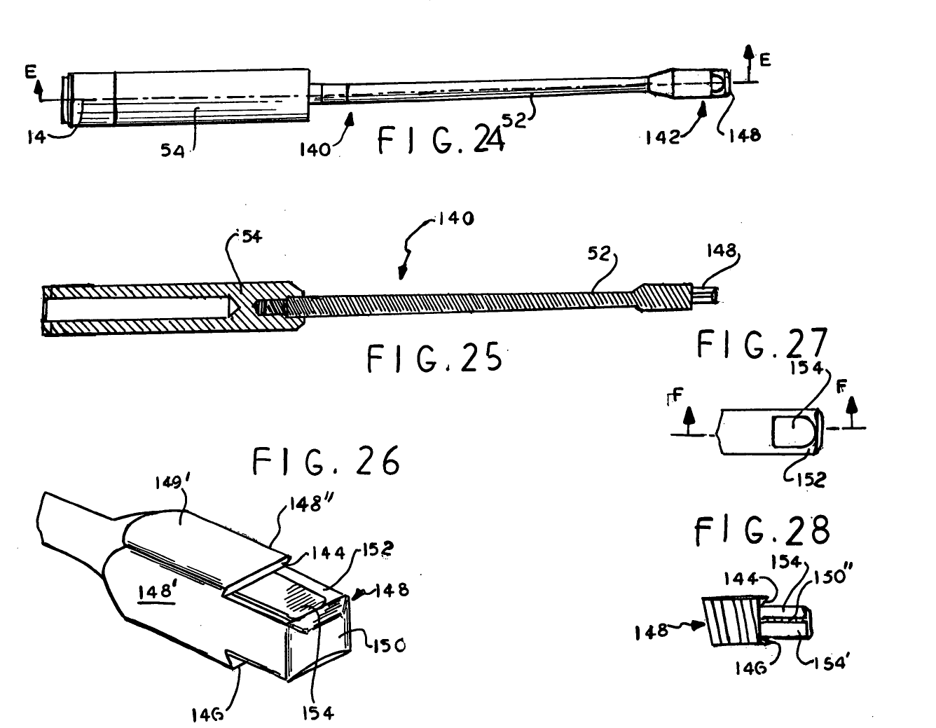

In another embodiment, an instrument 80 according to the present invention is shown in FIGS. 16-20. The shank 52 includes a handle 54 attached at a proximal end, and a cutting head 82 attached at a shank distal end as in the previous embodiment. The cutting head 82 in the embodiment shown in FIGS. 16-20, includes two transversely cantilevered cutting members 84, 86 terminating distally with convex cutting edges 88, 90. A guide member 92 extends from the side wall 68 between the cutting members 84, 86 and terminates in beveled non-cutting distal end 94. The cutting members 84, 86 extend from the cutting head 82 side wall 68 and are free at their opposite cantilevered ends spaced from the side wall 68. The channel shaped opening space between the members 84, 86 and side wall 68 provides a line of vision for a surgeon to observe the spinal area which is being worked on, as in the embodiment shown in FIGS. 7-11. The convex cutting edges 88, 90 provide a curved cutting edge against the vertebrae providing smoother cutting. The beveled distal portion 94 of the guide member 92 provides a tapered contact surface for slipping the guide member between adjacent vertebra. The convex cutting surfaces 88,90 of the cutting members 84, 86, for example, may also extend at various angles in relation to the side wall 68. The alternative cutting edges shown in FIGS. 9, 12, 9a, as well as FIGS. 18 and 21a, may also be positioned at various angles in relation to the side wall.

Another embodiment of a cutting head 100 according to the present invention is shown in FIGS. 21-23 and includes proximal convex cutting edges 88, 90 in cantilevered relation as shown in FIG. 18. However, planar guide member 102 terminates in a distal cutting edge 106 and includes side cutting edges 104 extending continuously from the edge 106 at a radius junction therebetween along both sides of the guide 102. The cutting edges, shown in FIGS. 21, 22, 23, enable the guide member to be used during surgery for cutting as well as guiding the proximal cutting edges 88, 90. The guide member cutting edge 106 and side cutting edges 104 are positioned between the cutting edges 88, 90 of the cutting members 84, 86 to cut away vertebrae and disc material between adjacent vertebrae. The side cutting edges 104 are closer to the longitudinal axis than the cutting edges 88, 90 of the cutting members 84, 86. The side cutting edge 104 may also be used to scrape away disc material and vertebrae material when the instrument is twisted about its longitudinal shank axis.

By way of example, during a surgical procedure the side cutting edges 104 make contact with disc material between adjacent vertebrae by which the guide member can cut and guide the proximal cutting edges 88 and 90 to the desired adjacent endplates or vertebrae. The thrust needed for cutting can be provided by the surgeon or a hammer applied to the proximal handle, or by other means such as a slap hammer.

In another embodiment, cutting head 110 is shown in FIG. 21a. The cutting head 110 is similar to the cutting head 100 shown in FIG. 21, however, the cutting edges 116, 118 are concave. The concave cutting edges 116, 118 provide a different angle of cutting along adjacent vertebrae. The cutting edges lie in a direction that is transverse to the longitudinal axis direction and are also beveled in a direct ion away from the axis.

Another embodiment of a cutting head is shown in FIGS. 21b-21d. The cutting head 110′ is similar to the cutting head 110 and includes a guide member 102 having upper and lower longitudinally extending upper and lower respective cutting edges 104, 104′ and distally facing concave cutting edges 116 and 118. However, the cutting members 113 are also arcuate and partially encircle axis 14″ in opposing mirror image curves toward each other, FIG. 21c. In FIG. 21d, the concave and arcuate cutting edges 116, 118 form arcuate shaped cuts 119′ in the adjacent vertebrae 119, 119″.

In another embodiment, an instrument 120 according to the present invention is shown in FIGS. 7a-7d. The shank 122 includes a handle 124 attached at a proximal end, and a cutting head 126 attached at a distal end. The cutting head 126 in the embodiment shown in FIGS. 7a-7d, includes two transversely cantilevered members 134, 136 terminating distally with linear transverse cutting edges 128, 130. A linear planar guide member 138 extends from a side wall 132 between the cutting edges 134, 136. The members 134, 136 are cantilevered from the side wall 132 and are free at their opposite transverse ends. The channel like opening between the members 134, 136 and side wall 132 provides a clear line of vision for a surgeon to observe the spinal area which is being worked on as in the previous embodiments. The members 134, 136 are more gradually sloped at side edges 134′ toward the head and shank from the cutting edges 128, 130, than in the prior embodiments, providing the members with more surface area than in the previous embodiment shown in FIG. 7.

In an alternative embodiment (not shown), the members 134, 136 shown in FIGS. 7a-7b can have a cutting edges along their generally longitudinally extending curved side edges 134′. The increased side wall surface area provides for a longer cutting edge than in the embodiment shown in FIG. 7. Further, a combination (not shown) of side wall cutting edges and guide member cutting edges may be provided for cutting at different portions of the adjacent vertebrae and disc material.

In another embodiment, an instrument 140 according to the present invention is shown in FIGS. 24-28. The shank 52 includes a handle 54 attached at a proximal end, and a cutting head 142 attached at a distal end as in the previous embodiment. The cutting head 142 in the embodiment shown in FIGS. 24-28, includes two linear and transverse cutting edges 144, 146 terminating distally of the head.

A guide member 148 extends from the head at and between side walls 148′ and 148″ and beneath the cutting edges 144, 146 and includes a distal end wall 150 traversing the longitudinal axis of the instrument and connecting the side walls to form a guide member having a generally rectangular shape. The guide member distal end surface is chamfered.

The guide member 148 includes a substantially longitudinally extending central openings 154, 154′ defined by the outer periphery of the guide member. The outer periphery has an upper surface 152 running along the perimeter of the guide member 148. The openings 154, 154′ form channels defined on a bottom surface by web 150′ extending transversely across the guide member as shown in FIG. 28. In an alternative embodiment, the openings may communicate with a substantially central proximal opening in the body 149′ of the cutting head, or in another alternative embodiment may extend through the guide member without a web.

In another embodiment, cutting head 160 is shown in FIG. 29. The cutting head 160 is similar to the cutting head 148 shown in FIG. 26, however, the upper periphery surface includes a cutting edge 162 extending along the perimeter defining the opening 154 in the guide member 164. The peripheral cutting edge 162 provides cutting to another portion of the adjacent vertebrae and spinal material under the proximal cutting edges 144, 146. The cutting edge 162 on the guide member is beneath and distal to the proximal cutting edges 144, 146, i.e., closer to the longitudinal axis 14 than edges 144,146. The distal cutting edge 162 extends about the guide member and may also be used to scrape material between the adjacent vertebrae by rotating the instrument and thereby the cutting head about the longitudinal axis 14.

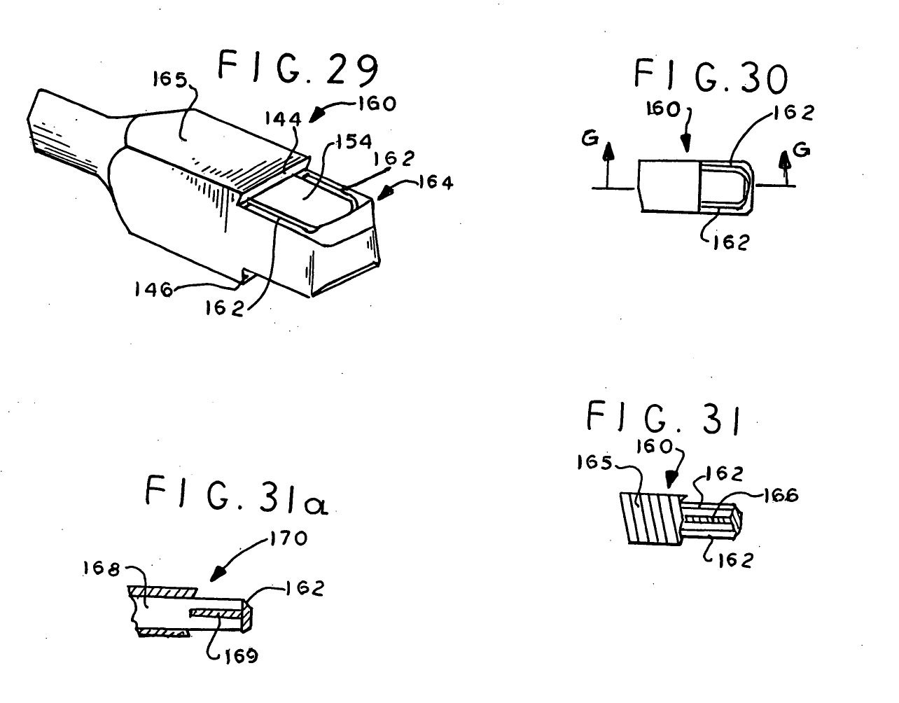

The guide member includes a web 166 connecting the sides of the guide member, as shown in FIG. 31 (cross section taken along line G-G in FIG. 30), and a solid body portion 165 of the cutting head 160. In an alternative embodiment shown in FIG. 31a (similar to cross section taken along line G-G in FIG. 30), the guide member opening 154 extends through the guide member hollow interior cavity 168. The body portion of the cutting head also defines a communicating opening (not shown) to the ambient atmosphere with the guide member openings.

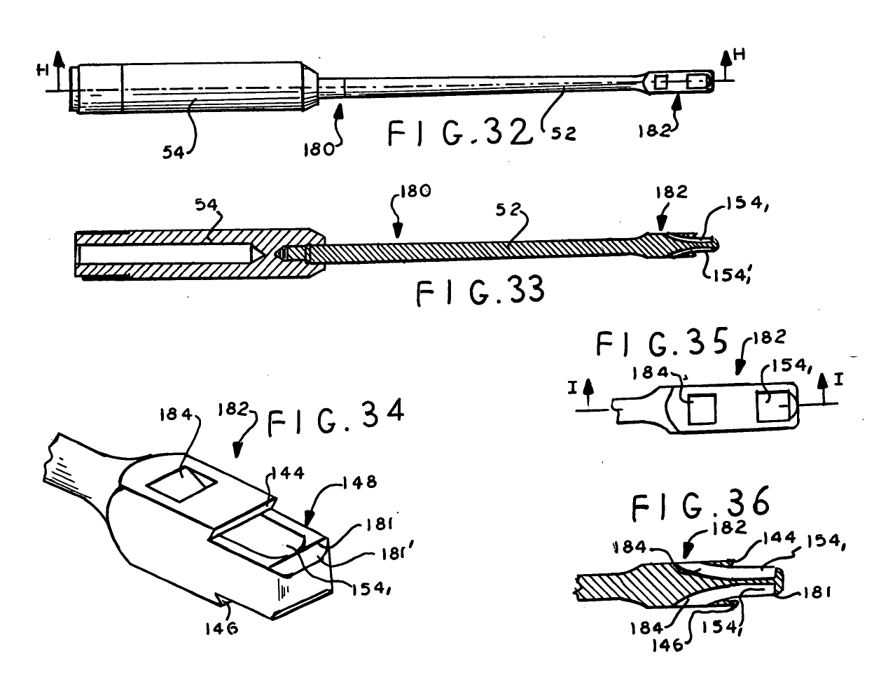

In another embodiment, instrument 180 according to the present invention is shown in FIGS. 32-36. The shank 52 includes a handle 54 attached at a proximal end, and a cutting head 182 attached at a distal end as in the previous embodiments. The cutting head 182 in the embodiment shown in FIGS. 32-36, is similar to the embodiment shown in FIGS. 24-28 and includes an opening 154₁ forming a channel and communicating with proximal opening 184 in the body of the cutting head 182. The openings form through channels (as shown in FIG. 36) from the distal opening 154 in the guide member 148 and the proximal opening 184 in the body of the cutting head 182. Both openings 184 and 154 are in spaced relation to each other in the top and bottom walls of the body of the cutting head 182 and the guide member 148, as shown in FIGS. 33-36. A distal transverse cutting edge 181 may be on the distal end of the guide member 148, and is adjacent a chamfered area 181′.

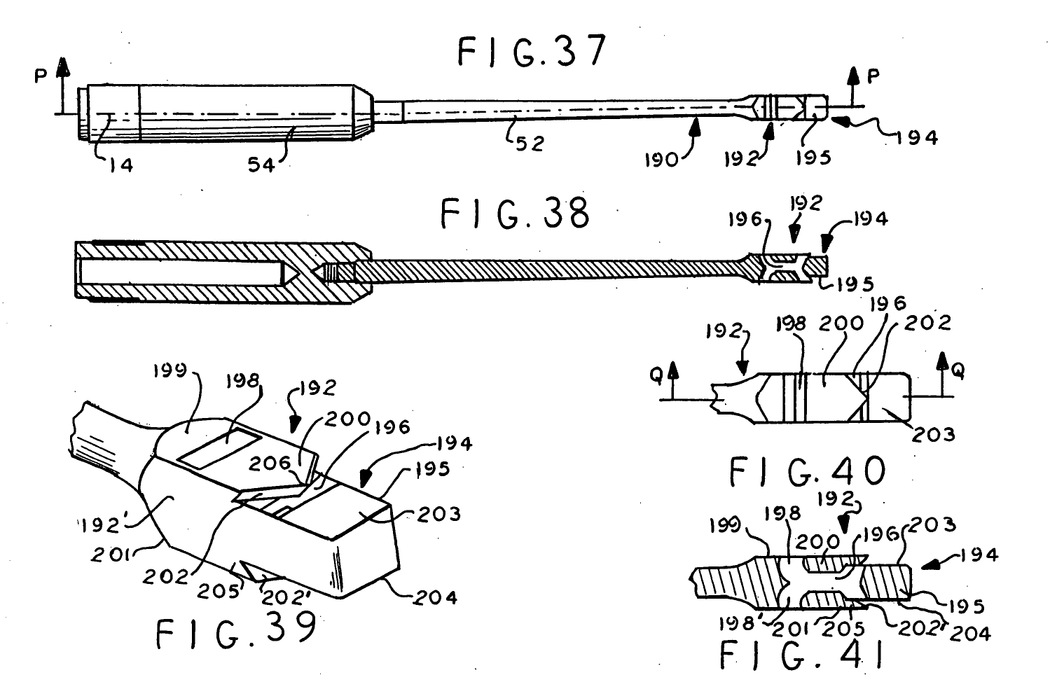

In another embodiment, instrument 190 according to the present invention is shown in FIGS. 37-41. The shank 52 includes a handle 54 attached at a proximal end, and a cutting head 192 attached at a distal end as in the previous embodiments. The cutting head 192 has a body 192′. A guide member 194 extends from the cutting head 192 body in the distal direction on and along the longitudinal along axis 14. The guide member 194 has a body 195. The cutting head body and guide member bodies have an internal chamber 196 which longitudinally extends through the bodies 192′ and 195, FIG. 41. The chamber 196 communicates with arcuate channel openings 198, 198′ terminating at the respective head body surfaces 199 and 201 at the proximal end of the head 192. The chamber 196 is in communication with an opening in the guide member top surface 203 and with an opening in the guide member bottom surface 204. The chamber 196 is in communication forms a longitudinal channel through the head body from the guide member to the head proximal region.

A V-shaped cutting edge 202 is formed on the top wall 205 distal end with the apex 206 facing in the distal direction. A second V-shaped cutting edge 202′ is formed on the bottom wall 205 of the cutting head with its apex facing in the distal direction. The cutting edges 202 and 202′ are juxtaposed relative to each other as best seen in FIG. 41. Cutting edges 202 and 202′ are each formed of two legs tapering towards one another and distally terminating at the apex 206.

In operation, bone chips from the cut vertebrae enter and are stored in the chamber 196 for removal later and/or also can pass out through the rear openings 198 during the cutting or later. The cutting edges form channels in the upper and lower vertebrae as the guide member guides the cutting edges to uniformly cut the two adjacent vertebrae. The guide member preferably is closely received between the upper and lower adjacent vertebrae.

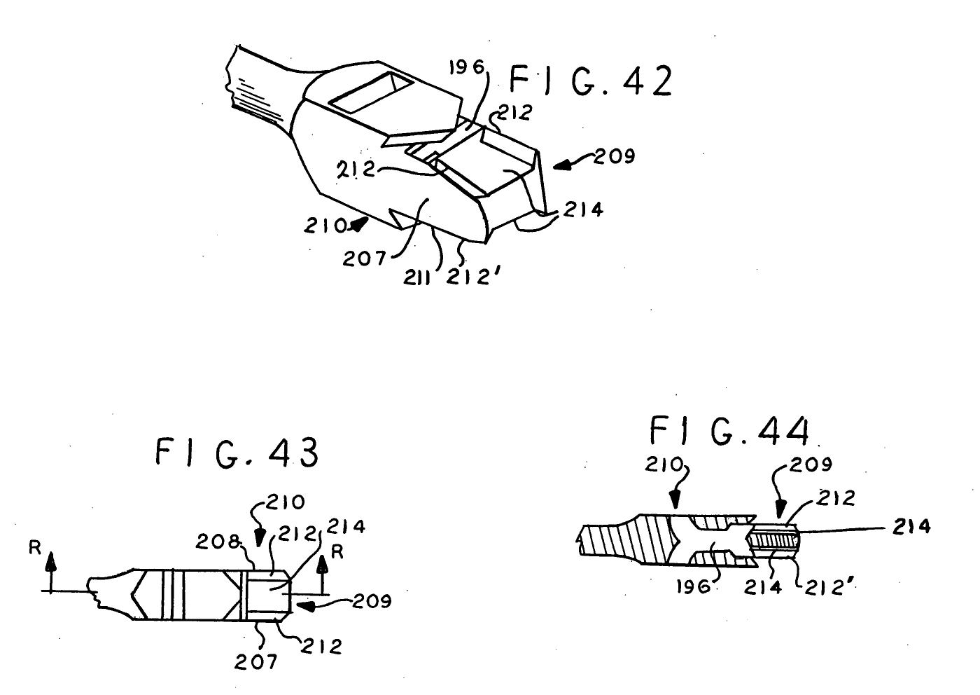

In another embodiment, cutting head 210 is shown in FIG. 42. The cutting head 210 is the same as the cutting head 192 shown in FIG. 39, except the guide member 209 body 211 includes cutting edges 212, 212′. These edges 212, 212′ are linear and extend along the guide member respective top and bottom side walls 207 and 208. In particular, the edges 212, 212′ are located at the longitudinally extending upper and lower longitudinally extending peripheries of the guide member 209. A recess 214 is formed in the guide member upper and lower surfaces between the edges 212 and 212′. The cutting edges 212, 212′ may be used to assist the V-shaped cutting edges in cutting channels in the upper and lower vertebrae and/or for scraping when the instrument is rotated about its longitudinal axis. The chamber 196 and its openings provide ingress and egress for the cut material fragments. To perform scraping, the guide member is inserted first into the disc space and rotated so that its edges 212, 212′ can scrape the vertebrae and/or disc material. Then, if desired, after the scraping, the cutting head is inserted further into the disc space to form the channels with the V-shaped cutting edges. The guide member is provided a length so as to perform the scraping for the desired depth into the disc space. The V-shaped upper and lower edges form a penetration depth limiting stop for the scraping function by abutting the vertebrae when only the guide member is inserted into the disc space. The body 211 of the guide member 209 may also include a channel which communicates with the chamber 196.

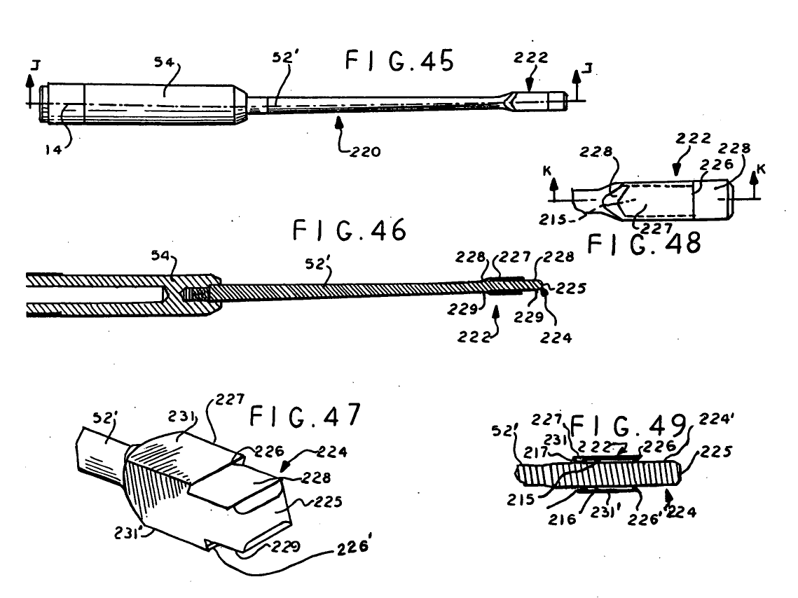

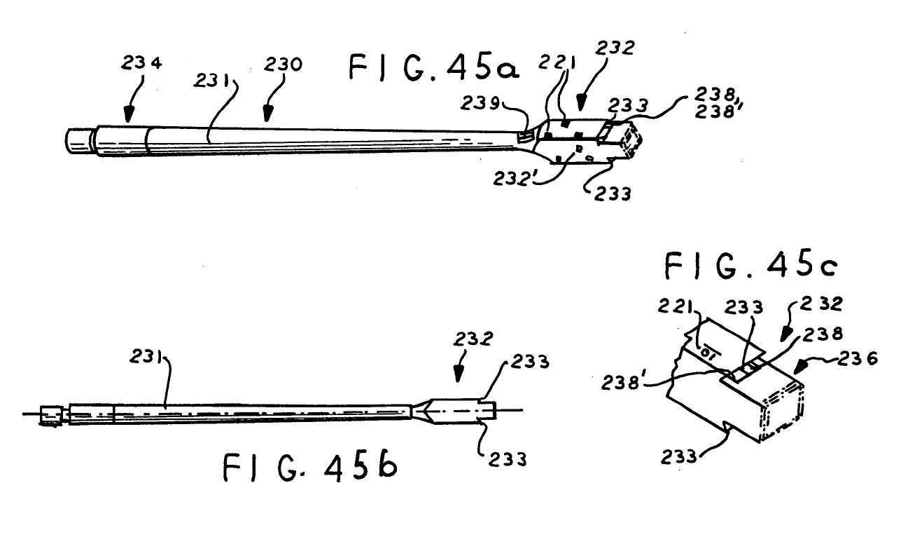

In a further embodiment, instrument 220 according to the present invention is shown in FIGS. 45-49. A shank 52′ includes a handle 54 attached at a proximal end, and a cutting head 222 is attached at the shank distal end. The cutting head 222 includes a guide member 224 having a body 224′ defining a generally rectangular structure with a blunt distal end 225. Upper and lower respective cutting edges 226, 226′ are distally disposed on the head body 227. The cutting edges are linear and transverse to the shank longitudinal axis 14. The cutting edges 226, 226′ meet adjacent vertebrae with the guide member 224 being inserted into the disc space between the adjacent vertebrae to guide the edges 226, 226′ to a uniform depth into the vertebrae.

The cutting head 222 has upper and lower longitudinal planar channels 215 and 216. The channels 215 and 216 have distal openings at the guide member 224 and proximal openings 217, 218 facing the shank 52. The guide member 224 has upper and lower respective planar surfaces 228, 229. These surfaces 228, 229 extend longitudinally through the head body 227 to form a respective side wall surface of the channels 215 and 216. The body 227 has a top wall 231 and a bottom wall 231′. These top and bottom walls form a longitudinal wall of the respective channels 215 and 216. As a result, cut material can pass through the channels 215 and 216, and thus through the cutting head body 227 in parallel planes.

In a further embodiment, FIGS. 45a-45c, an instrument 230 according to the present invention has a shank 231 with a handle 234 attached at the shank proximal end and a cutting head 232 attached at the shank distal end. The cutting head 232 has cutting edges 233 is similar to the cutting head shown in FIG. 47 of the previous embodiment, except a chamber 238 is in the body of the guide member 236. The chamber 238 extends through the guide member and is in communication with the top and bottom surfaces of the guide member via openings 238′ in the guide member and communicates with openings 239 in the proximal end of the cutting head 232, as shown in FIG. 45a similarly to chamber 196 in the cutting head 210 of FIG. 44. Depth of penetration indicia 221 are on the head top and bottom surfaces.

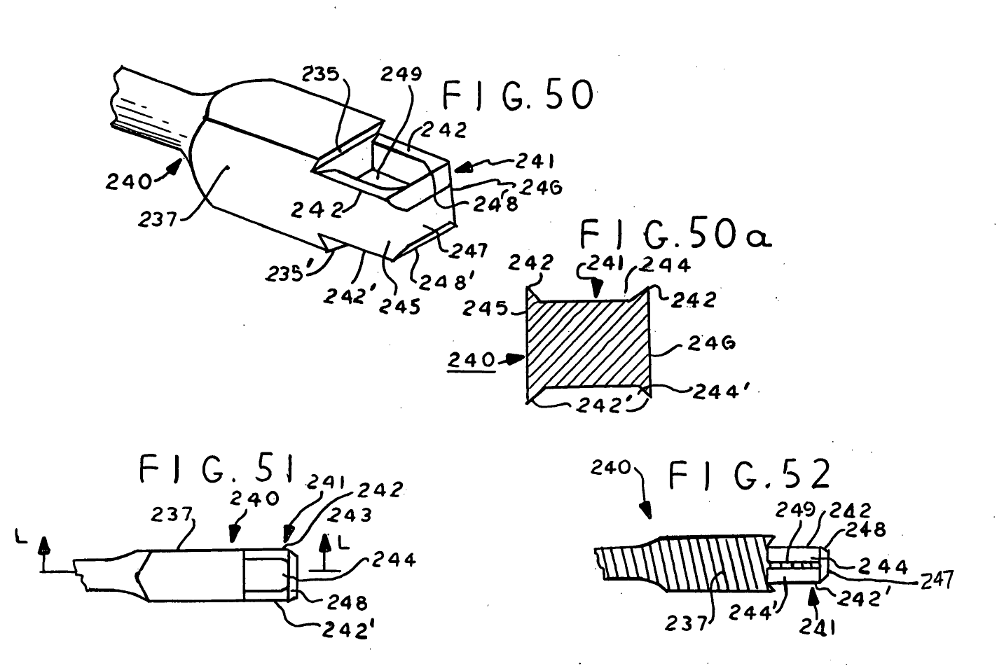

In another embodiment, cutting head 240 is shown in FIGS. 50-52. The cutting head 240 has a body 237. The body upper and lower walls terminate in transverse linear cutting edges 235, 235′. A guide member 241 extends distally from the body 237. The guide member 241 has side walls 245 and 246 and a distal end wall 247. The side walls and end wall each have contiguous upper and lower peripheral surfaces forming a U-shape. These surfaces are formed into two spaced upper and lower respective longitudinal cutting edges 242, 242′ extending along the longitudinal length of and at the sides of the guide member 240. The distal upper and lower transverse surfaces are formed into transverse distal edges 248, 248′ in communication with the respective longitudinal edges 242, 242′.

A transverse web 249 is between the upper and lower edges and is connected to the side walls 245, 246, distal end wall 247 and the head body 237 at its distal end wall 237′, FIGS. 50 and 52. An upper recess 244 is defined between the peripheral upper cutting edges 242 and 248 on the guide member 241. A lower recess 244′ is defined between the peripheral upper cutting edges 242 and 248 on the guide member 241. The distal cutting edge 242 may also be used to scrape material between the adjacent vertebrae by rotating the instrument and thereby the cutting head about the longitudinal axis 14. The recesses form bone debris receiving chambers.

In an alternative embodiment, the web 249 may be omitted in FIGS. 50 and 52 so that the two recesses 144 and 144′ form a single chamber for receiving cut bone debris.

In a still alternative embodiment, the head 240 of FIG. 50 may be constructed similarly to the head of the instrument in the embodiment of FIGS. 45-49.

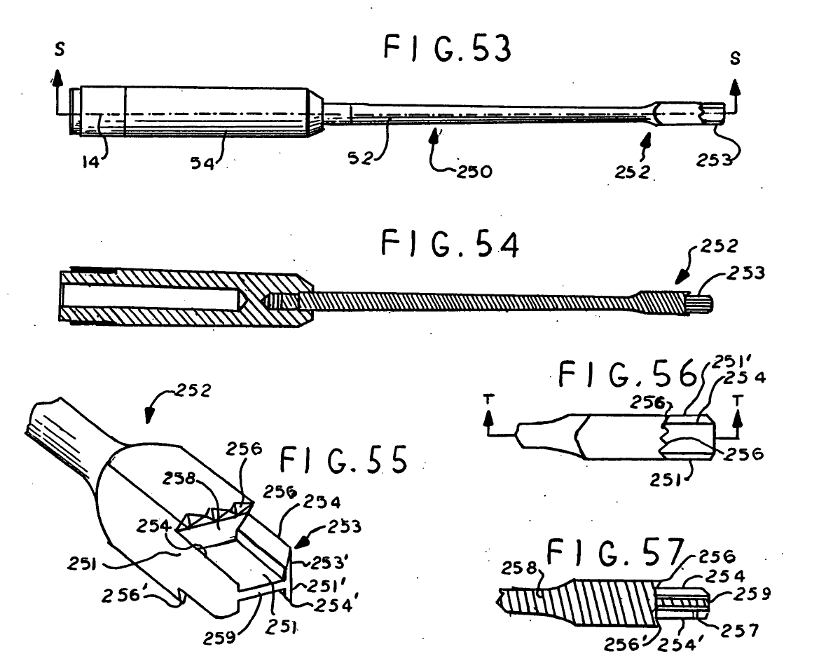

In another embodiment, an instrument 250 according to the present invention is shown in FIGS. 53-57. The shank 52 includes a handle 54 attached at a shank proximal end, and a cutting head 252 attached at the shank distal end as in the previous embodiments. The cutting head 252 includes a guide member 253 having a body 253′ with side walls 251, 251′ which are coextensive with the side walls of the head 252. The side walls 251 and 251′ terminate in upper longitudinal cutting edges 254 and lower longitudinal edges 254′, the edges being located along the upper and lower perimeters of the side walls. A web 257 extends medially between the upper and lower edges 254, 254′, respectively, from the cutting head body 258 to the distal end 259 of the guide member. The cutting edges 254, 254′ also form top and bottom vertebral guides for the guide member 253. While these edges cut into the vertebrae, they also center the head with respect to such vertebrae as they are inserted into the disc space and vertebrae defining that disc space.

Proximal upper and lower respective cutting edges 256, 256′ (the edges being proximal relative to the guide member distal edges 254, 254′) are preferably saw toothed or serrated as shown in FIG. 55, but may also be other configurations. The guide member distal cutting edges 254, 254′ are a lesser distance from the longitudinal axis than the saw toothed proximal cutting edges 256.

During spinal surgery, the saw toothed cutting edges 256 cut the adjacent vertebra while the lower and distal guide member cutting edges 254 cut the vertebrae and disc material and can also be used to scrape the disc space if the instrument is rotated about its longitudinal axis.

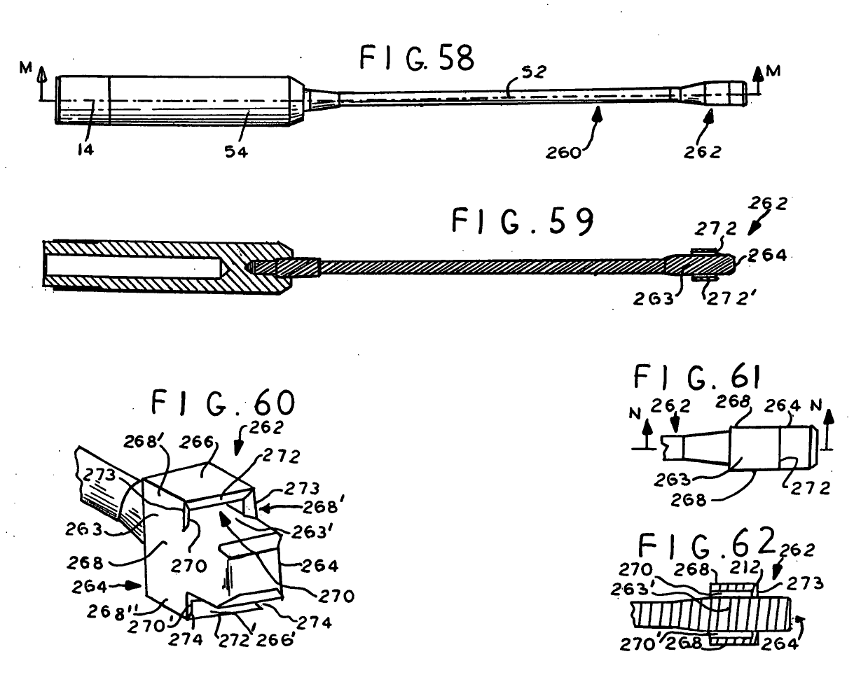

In another embodiment, an instrument 260 according to the present invention is shown in FIGS. 58-62. The shank 52 includes a handle 54 attached at a proximal end, and a cutting head 262 attached at a distal end as in the previous embodiments. The cutting head 262 includes a body 263 from which extends guide member 264. Member 264, FIG. 62, may be an extension of a central portion 263′ of the body 263 which in turn may be an extension of the shank 52. The cutting head 262 is a generally box-like structure having an interconnected guide member 264 extending distally from the central body portion 263′ of the head 262 along the longitudinal axis. The body 263 of the cutting head, FIG. 60, includes a top transverse wall 266 and a bottom transverse wall 266′ connected to two side walls 268. These walls terminate at proximal upper transverse edge 272 and lower transverse edge 272′. The side walls 268 have upper wall portions 268′ connected to top wall 266 and lower wall portions 268″ connected to bottom wall 266′. The top wall 266, side wall portions 268′ and central body portion 263′ define an upper channel 270 therebetween.

The channel 270 is open at its distal end at the guide member 264 and at its proximal end facing the shank 52. The bottom wall 266′, lower side wall portions 268″ and central body portion 263′ define a lower channel 270′ therebetween. The channels 270, 270′ are open at their distal ends at the guide member 264 and at their proximal ends facing the shank 52. These channels provide passages for material cut away from the vertebrae and disc material while the vertical and horizontal cutting edges 272 are cutting the adjacent vertebrae and adjacent disc area.

The upper side wall portions 268′ on opposite sides of the head 262 have cutting edges 273 that are normal to edges 272 and facing the guide member 264. Edges 272 and 273 form an upper rectangular cutting volume for cutting the upper vertebral material transversely and vertically. The lower side wall portions 268″ on opposite sides of the head 262 have cutting edges 274 that are normal to edge 272′ and facing the guide member 264. Edges 272′ and 274′ form a lower rectangular cutting volume for cutting the lower vertebral material transversely and vertically.

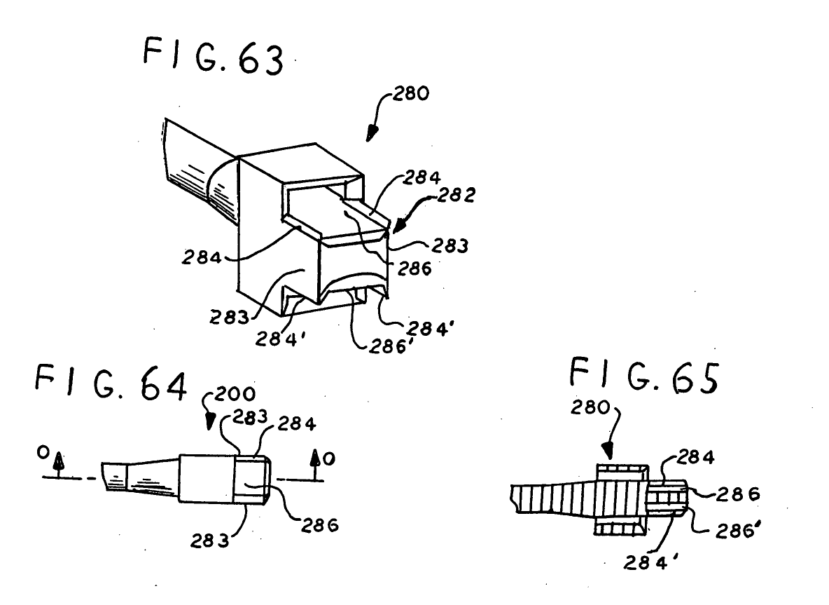

In another embodiment, a cutting head 280 is shown in FIGS. 63-65. The cutting head 280 is the same as the cutting head 262 shown in FIG. 60, except the guide member 282 longitudinal side walls 283 upper and lower peripheral edge surfaces each includes a longitudinal respective upwardly or downwardly facing cutting edge 284, 284′ extending along the longitudinal side upper and lower perimeters of the guide member 282. Upper and lower recesses 286, 286′ is defined between the periphery side wall cutting edges 284 on the guide member 282. The distal cutting edges 284, 284′ on the guide member may also be used to scrape material between the adjacent vertebrae by rotating the instrument and thereby the cutting head about the longitudinal axis 14.

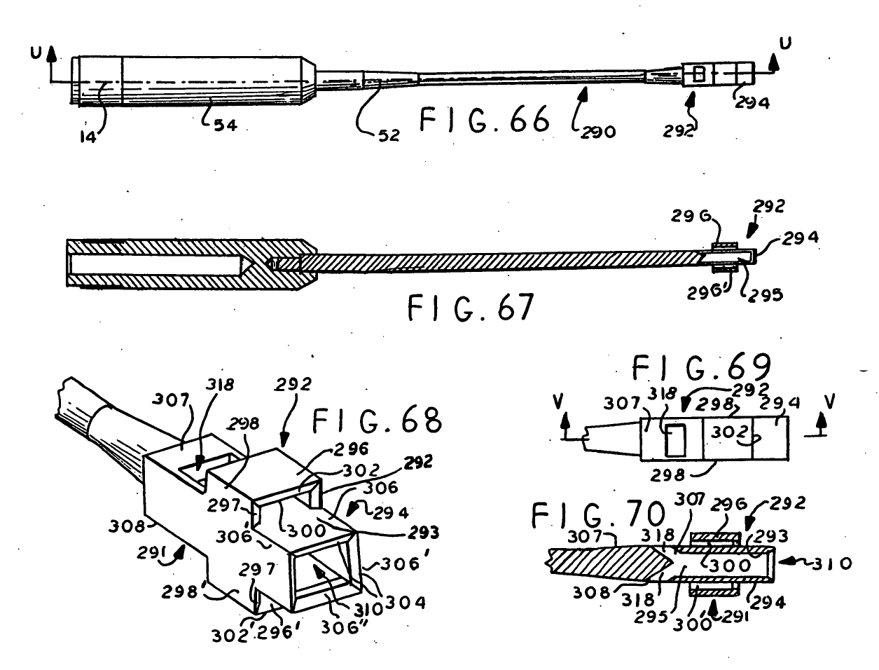

In another embodiment, an instrument 290 according to the present invention is shown in FIGS. 66-70. The shank 52 includes a handle 54 attached at a proximal end, and a cutting head 292 attached at a distal end as in the previous embodiments. The cutting head 292 includes a distally extending guide member 294. The body 291 of the cutting head includes a central member 293 having a longitudinal channel 295. The channel 295 has a distal opening 310 at the distal end of the guide member 294 and proximal openings 318 in the form of arcuate channels in communication with the channel 295. The openings terminate in the respective top and bottom surfaces 307 and 308 of the body central member 293 at the proximal end at the shank 52 end of the head 292. The body 293 includes a top wall 296 connecting two upper side wall portions 298 in spaced relation to each other at the top of the cutting head. Wall 296 and portions 298 and member 293 define an upper rectangular longitudinal channel 300 between the top wall 266 and the central member 293.

The body 293 includes a bottom wall 296′ connecting two lower side wall portions 298′ in spaced relation to each other at the bottom of the cutting head. Wall 296′ and portions 298′ and central member 293 define a lower rectangular longitudinal channel 300′ between the bottom wall 296′ and the central member 293. The distal ends of the top wall 296 and bottom wall 296′ terminate at transverse linear cutting edges 302 and 302′, respectively. The distal ends of the upper and lower side wall portions terminate in vertical cutting edges 297.

The guide member 294 preferably includes four walls comprising top wall 306 and bottom wall 306″ and side walls 306′. These walls are an extension of the body central member 293 and are coplanar therewith. These walls define an opening 310 therebetween to the channel 295 and terminate in distal cutting edges 304. The resulting box-like cutting edges 304 are beneath the upper proximal cutting edge 302 and above the lower proximal edge 302′ and are a lesser distance from the longitudinal axis 14 than the proximal edges.

The box-like cutting edges 304 cut away at the disc material between the vertebrae while the proximal cutting edges 302, 302′ cut the vertebrae, FIG. 70. The spinal debris cut by the proximal edges is channeled through the channels 300 and 300′ and debris cut by the guide member distal edges 304 is channeled into the end of the guide member via opening 310. The debris at opening 310 is passed through channel 295 which communicates with proximal openings 318 in the cutting head 292.

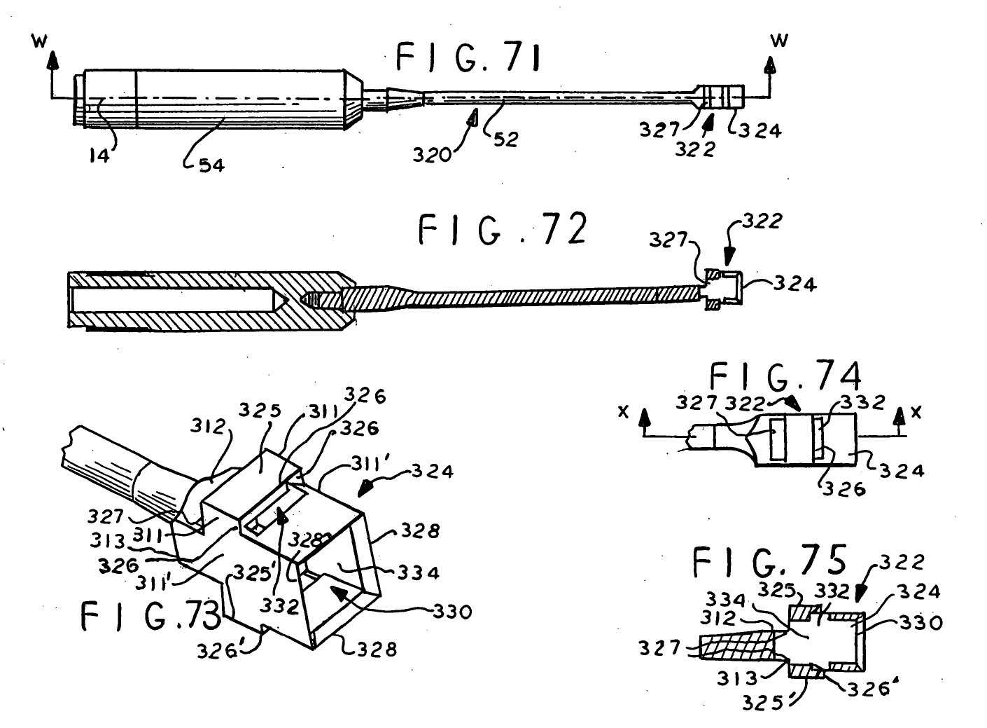

In another embodiment, an instrument 320 according to the present invention is shown in FIGS. 71-75. The shank 52 includes a handle 54 attached at a proximal end, and a cutting head 322 attached at a distal end as in the previous embodiments. The cutting head 322 includes a guide member 324 extending distally and integral with the body of the cutting head. Proximal cutting edges 326, 326′ are formed in top wall 325 and bottom wall 325′. Two opposite upper side wall portions 311 distally terminate in cutting edges 326. Similar edges are formed in bottom side wall portions. The top wall, bottom wall and side walls form a chamber 334. The head body side walls 311′ form the side walls of the guide member 324 as well as the upper and lower side wall portions 311.

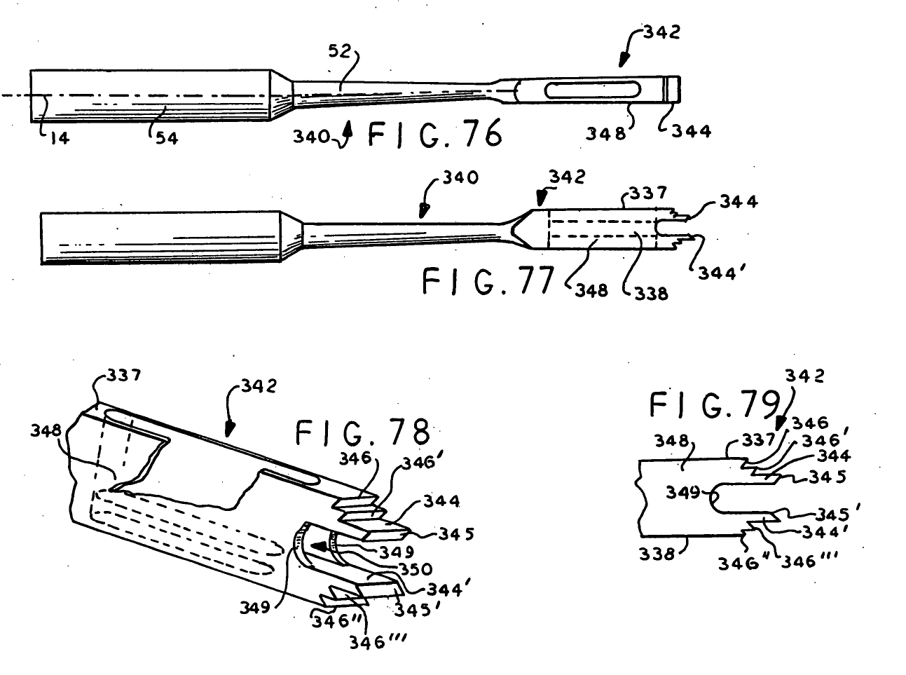

The guide member 324 terminates at four quadrilateral oriented distal cutting edges 328 defining opening 330 which communicates with a proximal opening 332 in the guide member at the edges 326 via longitudinal chamber 334. Proximal openings 327 are formed in top and bottom surfaces 311 and 312 of the cutting head and communicate with chamber 334, FIGS. 74-75. The openings 327, 330, and 332, and chamber 334, in concert provide egress for material cut away by the proximal and distal cutting edges 326, 326′, and 328, respectively.

The guide member 324 distally terminates with cutting edges 328 which define the distal end of the opening 330. The guide member cutting edges 328 are positioned closer to the longitudinal axis than the proximal cutting edges 326, 326′ and thus, are positioned to cut away disc material from between adjacent vertebrae while the proximal cutting edges 326 cut material from the adjacent vertebrae.

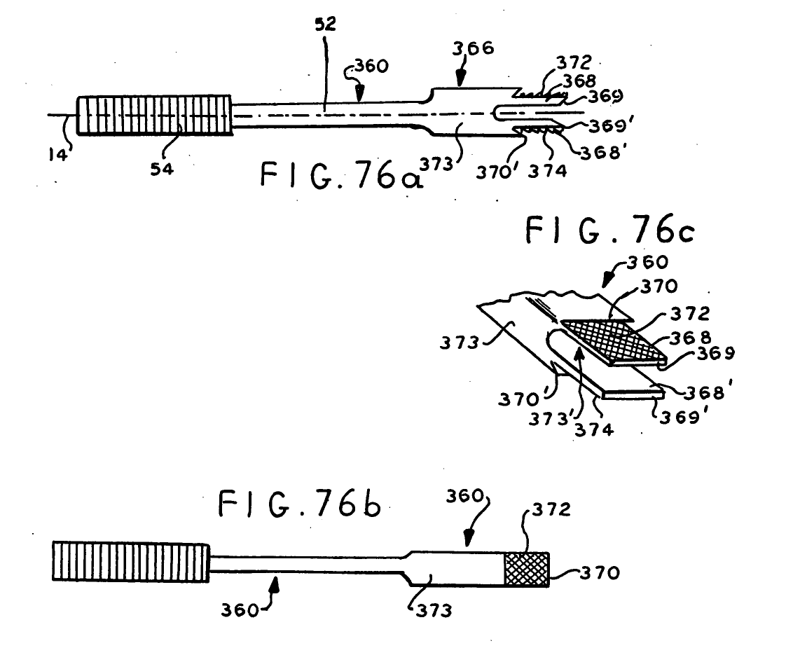

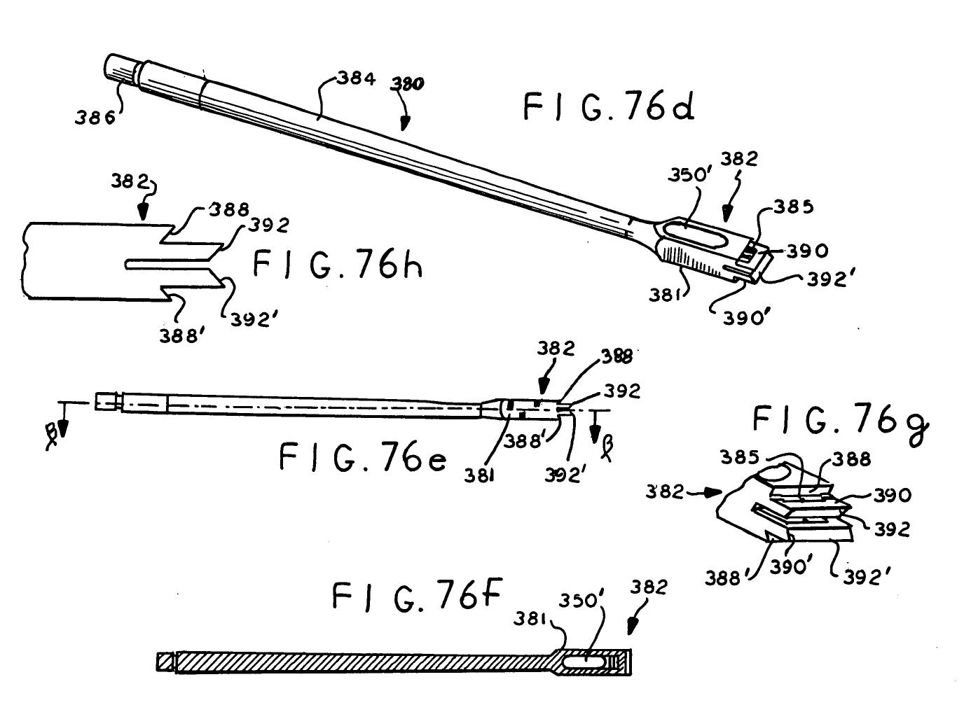

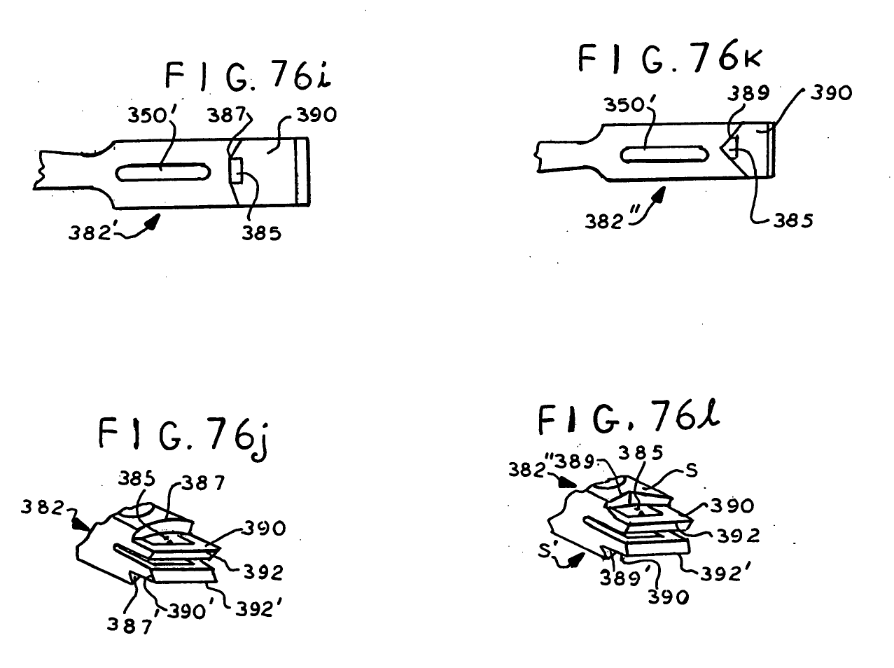

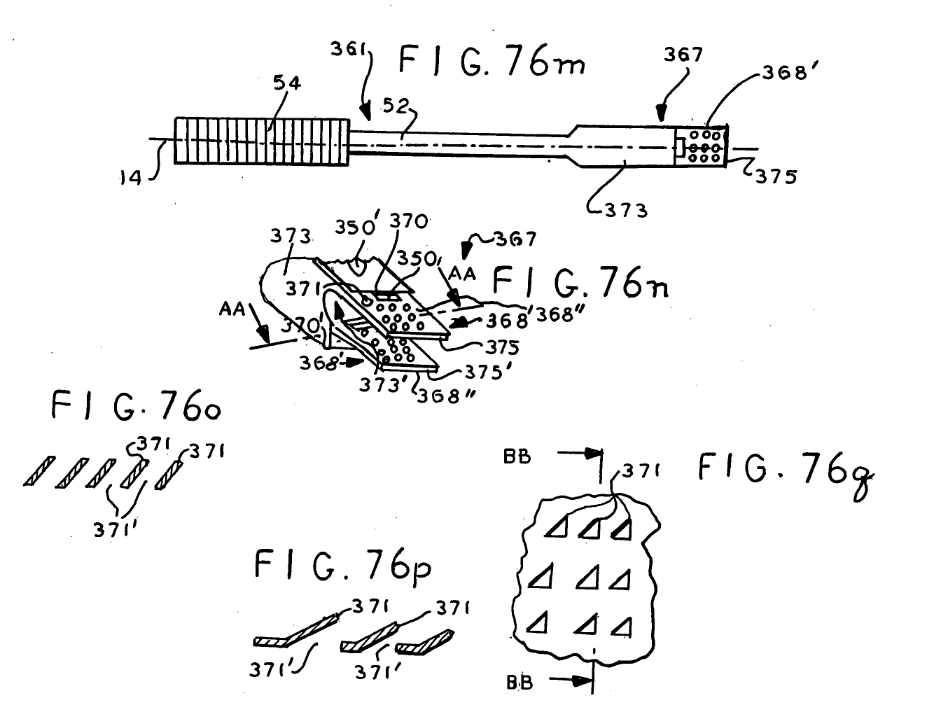

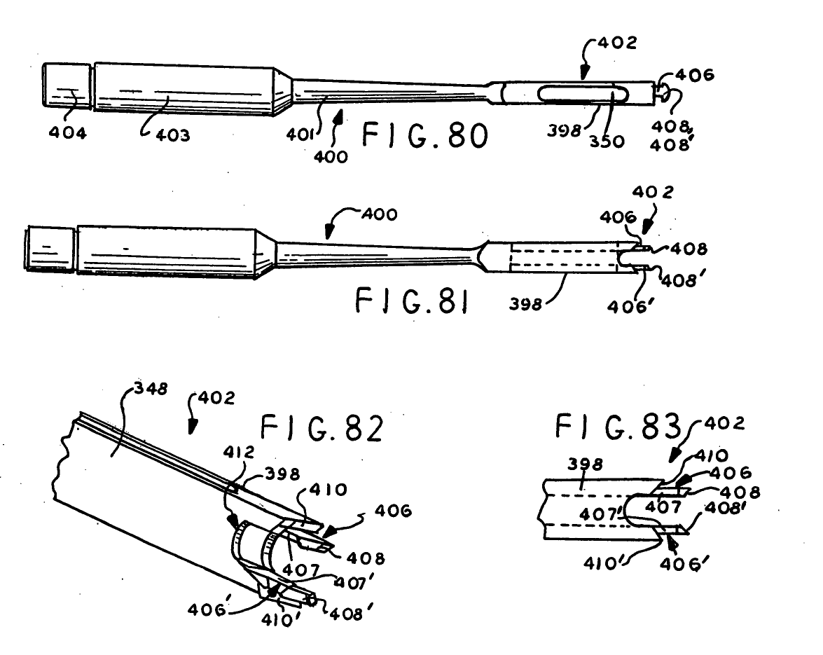

In another embodiment, an instrument 340 according to the present invention is shown in FIGS. 76-79. The shank 52 includes a handle 54 attached at a proximal end, and a cutting head 342 attached at a distal end as in the previous embodiments. The cutting head 342 includes a pair of spaced mirror image guide members 344, 344′ extending distally and terminating with respective distal cutting edges 345, 345′. Representative guide member 344 is integral with the body of the cutting head. Representative proximal cutting edges 346, 346′ are in mirror image relation with a like set of edges 346″ and 346′″ adjacent to member 344′ which also extend integrally with the body of the cutting head 342. The edges 346 and 346′ are in stepped relation to each other with edge 346′ slightly more distal than edge 346 whereas the edge 345 is significantly more distal than the stepped edges.

The body 348 of the cutting head 342 defines a substantially central longitudinal opening 350 extending through the top surface 337 and bottom surface 338 of the body 348. the opening extends through and to the head distal end between the distally extending guide members 344, 344′. Arcuate side walls 349 define the sides of the distal mouth of the opening 350 which mouth is more proximal than the stepped cutting edges. The opening 350 receives debris produced by the cutting edges during the spinal procedure.

The guide members 344 and 344′ respective cutting edges 345, 345′ cut disc material from adjacent vertebrae while guiding the proximate cutting edges to meet and cut from adjacent vertebrae. The opening 350 is adapted and positioned to facilitate cutting debris removal from the cutting site to the opening proximate the cutting edges. The guide members 344 and 344′ may also include an opening which communicates with the opening 350, as depicted in the embodiment of a cutting head shown in FIGS. 76d, 76g.

In another embodiment, an instrument 360 according to the present invention is shown in FIGS. 76a-76c and is similar to the previous instrument shown in FIGS. 76-79 except there is but a single upper and lower proximal edge 370 rather than a pair of stepped pair of edges and there is no central longitudinal chamber in the head body. The shank 52 includes a handle 54 attached at a proximal end, and a cutting head 366 is attached at a distal end as in the previous embodiments. The cutting head 366 includes a spaced pair of mirror image guide members 368, 368′ extending distally and terminating with a distal cutting edges 369, 369′. The guide members 368, 368′ are integral and one piece with the body 373 of the cutting head 366. Proximal upper and lower respective transverse linear cutting edges 370, 370′ extend from the body of the cutting head 360.

In the embodiment shown in FIGS. 76a-76c, the top surface of the guide member 368 includes a rasp 372, and in an alternative embodiment may also include a broach. Further, the rasp may be positioned at different locations on the cutting head as an alternative to the preferred positioning on the top surface of the guide member. The rasp surface shown in FIGS. 76a-76c preferably includes a plurality of sharp protuberances or raised projections such as pyramidal, conical or other rasp projections as known in this art and may comprise for example an array of saw teeth. A similar rasp surface is on the under surface 374 of the guide member 369′ in mirror image relation to surface 372. The rasp surface 372 preferably thus is on the top and bottom opposite surfaces of both of the guide members 368 and 368′ and may be used to scrape disc material and bone from adjacent vertebrae.

In another embodiment, an instrument 380 according to the present invention is shown in FIGS. 76d-76h and is similar to a previous instrument shown in FIGS. 76-79 except there are no rasp surfaces and there is a longitudinal chamber through the head. The shank 384 includes a handle 386 attached at a proximal end, and a cutting head 382 is attached at a distal end as in the previous embodiments. The cutting head 382 includes a pair of guide members 390, 390′ extending distally and terminating with respective distal cutting edges 392, 392′. The guide members 390, 390′ are integral and one piece with the body of the cutting head. As in this and the other embodiments, the body may be a separate element that is attached by threads and the like to the shank. Proximal linear and transverse cutting edges 388, 388′ also extend integrally with the body of the cutting head 382. The body 381 of the cutting head 382 defines a longitudinally extending elongated substantially central opening 350 extending therethrough.

In FIGS. 76d-76h, the top surfaces of the guide member 390, 390′ are each formed with an opening 385 (one being shown) extending therethrough and communicating with elongated longitudinally extending central opening 350′ (similar to the arrangement of openings shown in the embodiment of FIG. 78). The opening 385 on each guide member 390, 390′ is positioned adjacent to and beneath the proximal respective cutting edges 388,388′, and readily receives debris from cutting, and allows the debris to egress through the opening 350′ toward the proximal end of the cutting head 382.

Another embodiment of a cutting head is shown in FIGS. 76i and 76j. The cutting head 382′ includes concave curved proximal upper and lower distally facing cutting edges 387 and 387′. The cutting head 382′ includes a longitudinally extending chamber or opening 350′ therethrough and a spaced pair of opposing mirror image flat distally extending guide members 390, 390′ terminating in transverse linear distal respective cutting edges 392 and 392′. A chamber or opening 385 as in the cutting head 382, FIGS. 76i and j. The curved proximal cutting edges 387 and 387′ cause cut bone material to move toward the center of the cutting head and through the openings 385 in the upper and lower guide members 390, 390′ as the chisel is advanced into the vertebrae.

Another embodiment of a cutting head is shown in FIGS. 76k-76l. The cutting head 382″ includes upper and lower distally facing proximal V-shaped cutting edges 389 and 389′ with the apex of the “V” located more toward the proximal end of the cutting head at the shank. The cutting head 382″ includes a chamber or opening 350′. Guide members 390 and 390′ terminate in distal cutting edges 392 and 392′. An opening 385 is in the cutting head and is in the guide members in communication with the chamber 350′, FIGS. 76k and 761. The V-shaped cutting edges 389 and 389′ cause cut material to move toward the center of the cutting head and through the opening 385 in the guide members 390, 390′ into the chamber 350′ and exit the head via the chamber 350′ at the head top and bottom surfaces S and S′ in similar fashion as the openings and chamber in others of the embodiments discussed herein.

In another embodiment, an instrument 361 is shown in FIGS. 76m-76q. The shank 52 includes a handle 54 attached at a proximal end, and a cutting head 367 attached at a distal end as in the previous embodiments. The cutting head 367 includes a pair of like planar spaced mirror image guide members 368′ extending distally from the head and terminating at respective distal linear transverse cutting edges 375, 375′. The head further includes a longitudinally extending substantially central through opening 350′ as in the previous embodiments. Similar to the cutting head 360 shown in FIG. 76c, representative guide member 368′ is integral and one piece with the body 373 of the cutting head 367. Representative transverse linear upper proximal cutting edge 370 is in mirror image relation with a like lower edge 370′.

The body 373 of the cutting head 367 defines the substantially central longitudinal chamber or opening 350′ extending through a top surface and bottom surface of the body 373. The chamber or opening 350′ extends through and in communication with the head distal end between the distally extending guide members 368′ at opening 373′. The opening 350′ receives debris produced by the cutting edges during the spinal procedure via opening 373′ and openings 371 in the guide members. Openings 350′ extend through the guide members and are juxtaposed with and extending somewhat distally beyond the edges 370, 370′.

The upper and lower guide members 368′ each include a plurality of raised cutting edges 371 on their respective upper and lower surfaces 368″. The raised cutting edges 371 are shown in greater detail in FIGS. 76o and 76q which depict the upwardly extending cutting edges 371 and their corresponding openings 371′ beneath the cutting edges and which openings extend through the guide members. Preferably, the head body 373 further includes a substantially central opening 373′ at the body distal end communicating with the opening 350′ and communicating with the space between and defined by the opposing guide members 368′. The openings 371′ in the guide members communicate with the opening 373′ extending through the body of the cutting head and communicating with opening 350′ to facilitate the removal of cut debris from the cutting sites. The raised cutting edges 371 in a rasp like manner and further cut material from adjacent vertebrae while the guide members 368′ guide the proximal cutting edges 370 and 370′ to meet and cut from adjacent vertebrae.

In another embodiment, an instrument 400 according to the present invention is shown in FIGS. 80-83 and has a similar body 348 and opening such as opening 350′ as the embodiment shown in FIGS. 76-79. The shank 401 includes a handle 403 having an impact area 404 at a proximal end, and a cutting head 402 attached at a distal end. The impact area 404 is adapted to receive color coated rings for identification of the instrument by the surgeon. The cutting head 402 includes upper and lower mirror image guide members 406, 406′ extending distally and one piece integral with the body 398 of the cutting head 402. The head distally terminates in proximal upper and lower respective transverse linear cutting edges 410 and 410′, FIGS. 82 and 83.

The guide members 406, 406′ preferably distally terminate with arcuate convex juxtaposed cutting edges 408, 408′. In the alternative, the cutting edges 408, 408′ may include two legs distally converging at an apex to form a V-shaped cutting edge (not shown). The guide members each have a respective stem 407, 407′ of smaller transverse width than the transverse width of the cutting edges 408, 408′ to form a generally T or arrow-head shape with the wider transverse edges 408, 408′. The stem 407, 407′ allows cut material to fall around it during chiseling, thereby facilitating the removal of debris from the cutting site.

The upper and lower cutting edges 408, 408′ are in spaced relation to each other and are closer to the longitudinal axis than the proximal cutting edges 410, 410′. The transverse arcuate shaped cutting edges 408, 408′ provide different gradual cutting in the disc material than a transverse straight linear edge. The cutting head function is a substantially same manner as described for the instrument in FIGS. 71-79. The guide members 406, 406′ define a U-shaped space therebetween with the adjacent head portion as shown in FIGS. 85 and 86.

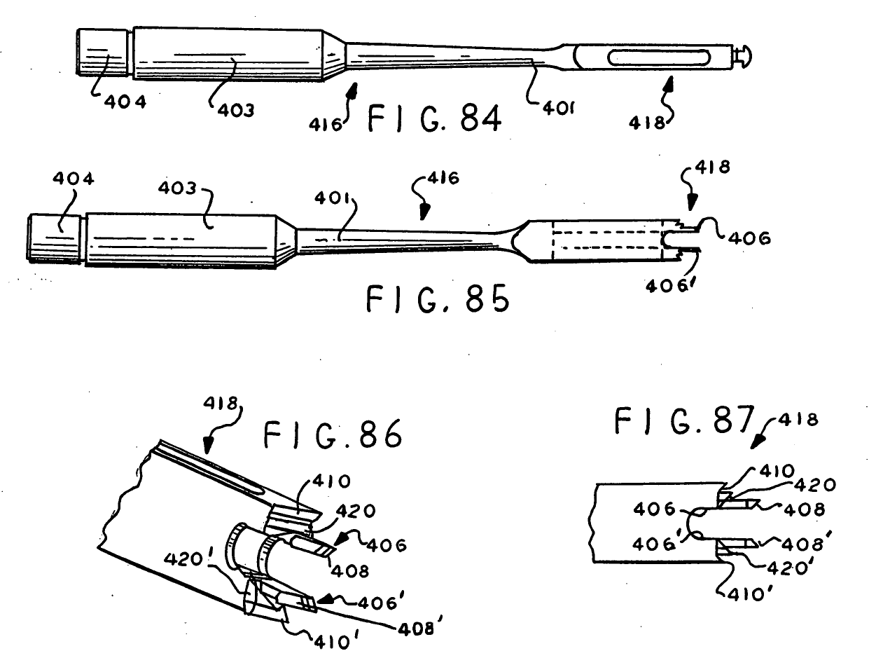

In another embodiment, an instrument 416 according to the present invention is shown in FIGS. 84-87 and includes similar structure as in the previous embodiments shown in FIGS. 76-79 showing stepped cutting edges and FIGS. 80-83 showing T shaped guide members with arcuate cutting edges. However, the cutting head 418 includes upper and lower respective transverse linear cutting edges 410, 410′ and intermediate V-shaped stepped cutting edges 420, 420′ culminating in a distal apex. The intermediate stepped V-shaped cutting edges are between the upper linear edge and upper guide member 406 (which is described in the embodiment of FIGS. 80-83) and between the lower linear transverse edge and the lower guide member 406′.

In FIGS. 76-79 the intermediate stepped cutting edges between the upper linear edge and upper guide member and between the lower edge and the lower guide member are linear, rather than V-shaped.

The guide members 406, 406′ also include the arcuate or arrow-head shaped cutting edges 408, 408′ as described in the previous embodiments. The proximal cutting edges 410, 410′ and the V-shaped edges 420, 420′ provide increased cutting at the vertebrae and at two different angles, i.e., the transverse cutting edges 410, 410′ and the angled cutting edges 420, 420′.

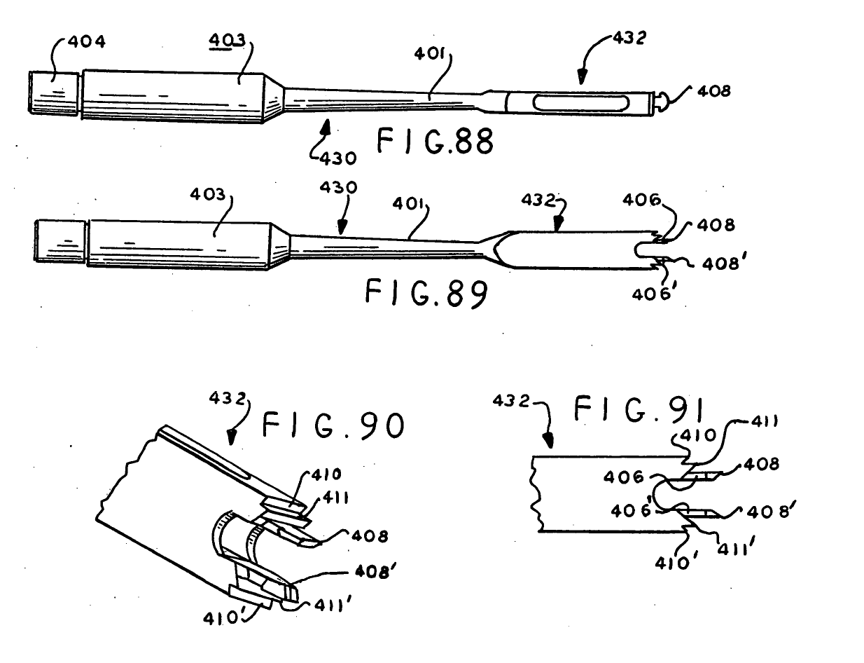

In another embodiment, an instrument 430 according to the present invention is shown in FIGS. 88-91 and includes similar stepped edges as in the previous embodiment of FIGS. 76-79 and a pair of guide members each terminating with arcuate edges as shown in the embodiments of FIGS. 80-87. The cutting head 432 includes two upper transverse linear stepped cutting edges 410, 411, and two mirror image lower transverse linear stepped cutting edges 410′, 411′.

A pair of mirror image spaced guide members 406, 406′ terminate in distal arcuate or arrow-head shaped cutting edges 408, 408′, both in spaced relation on the top and bottom of the cutting head. The proximate cutting edges provide two upper transverse cutting edges 410, 411 and two lower transverse cutting edges 410′, 411′ for increased cutting action at the vertebrae at different locations at the vertebrae. Further, the multiple cutting edges 410, 410′, 411, 411′ provide a first contact cutting edge 411, 411′ at a less distance from the longitudinal axis of the instrument than the second contact cutting edge 410, 410′ which cutting edged are at a greater distance from the longitudinal axis. Thus, cutting occurs staggered in time as well as in regions of the vertebrae.

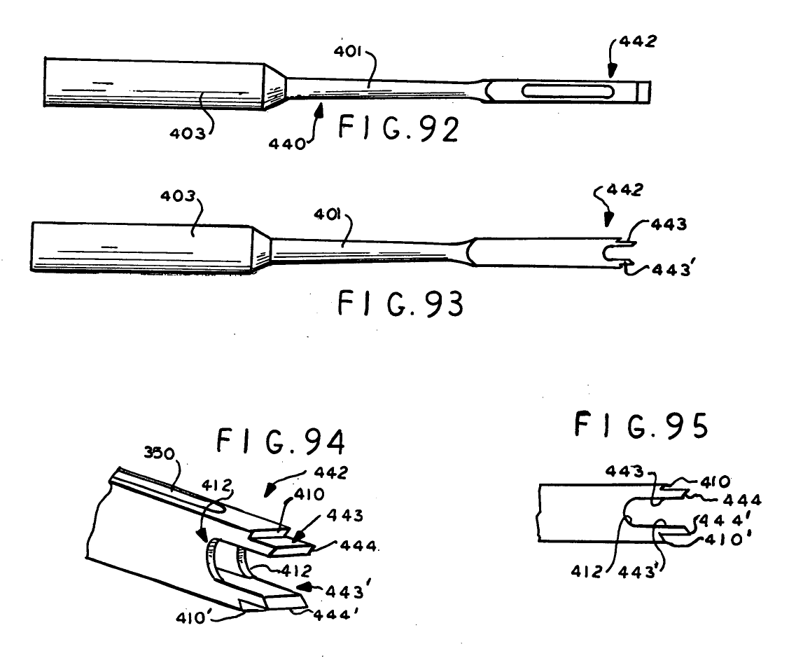

In another embodiment, an instrument 440 according to the present invention is shown in FIGS. 92-95 and includes similar structure as in the previous embodiments shown in FIGS. 88-91. However, there is no intermediate stepped cutting edge 411 as in the other embodiment, the cutting head 442 only includes upper and lower transverse linear proximal cutting edges 410, 410′ and a transverse linear distal cutting edge 444, 444′ terminating at each of the mirror image guide members 443, 443′ distal ends. Longitudinal opening 350 terminates in the space between the guide members 443 and 443′ as in certain of the prior embodiments. The head terminates in semi-circular edges 412 between the guide members.

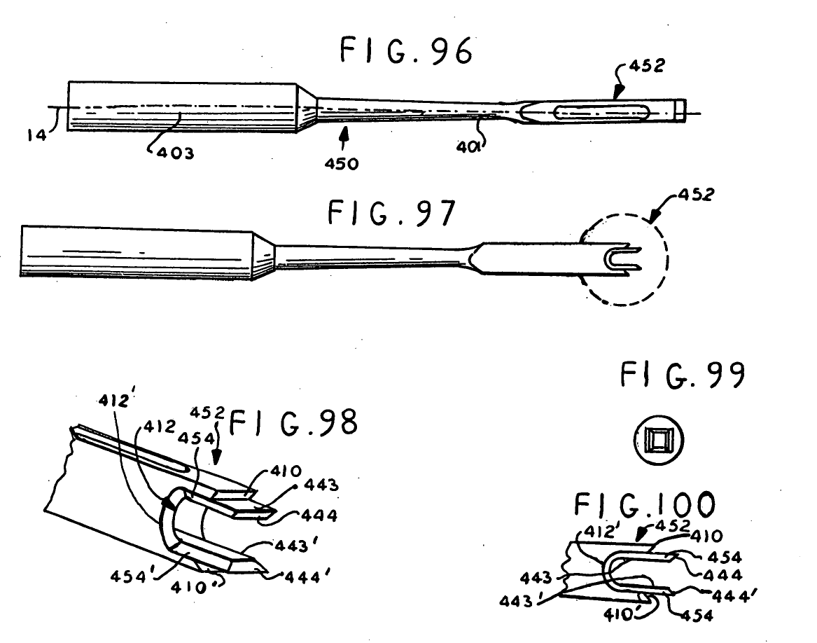

In another embodiment, an instrument 450 according to the present invention is shown in FIGS. 96-100 and includes similar structure as in the previous embodiments shown in FIGS. 92-95, including cutting edges 410, 410′ and 444, 444′. However, guide members 443 and 443′ and the intermediate arcuate side edge 412′ of the cutting head 442 includes a continuous side cutting edge 454 extending along the guide members 443, 443′ peripheral edges and along the arcuate edge 412′. Edge 454 is contiguous with the peripheral edges of the guide members 443, 443′ forming a U-shaped side edge that is both vertical between the guide members at edge 412′ and longitudinal at the edges of the members 443, 443′. The side cutting edges 454 forms a generally U-shaped cutting edge on opposite sides of the cutting head and provides an edge lying in a plane that is generally perpendicular to the plane of the distal and proximate cutting edges 444 and 410. The side cutting edges 454 can be used to scrape disc material and vertebrae bone when twisting the instrument about the longitudinal axis 14.

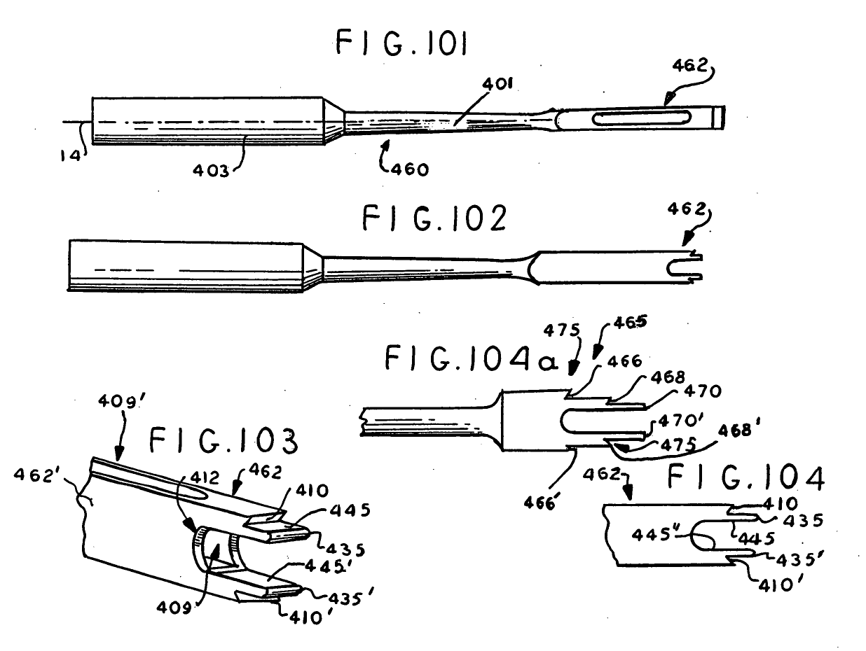

In another embodiment, an instrument 460 according to the present invention is shown in FIGS. 101-104 and includes similar structure as in the previous embodiments shown in FIGS. 96-100, i.e., the cutting edges 410, 410′. However, the mirror image spaced planar relatively thin guide members 445, 445′ of the cutting head 462 each terminates in a rounded non-cutting distal linear end edge 435, 435′ transverse to the longitudinal axis. The guide members are inserted into the disc space and bear against the vertebrae to provide guidance to the cutting edges 410, 410′ when cutting adjacent vertebrae. In an alternative, the guide members 445, 445′ may include an opening (not shown), similar to the opening 385 shown in FIG. 761, extending therethrough. The opening in the guide member 445 may communicate with the opening 409 extending through the body 462′ of the cutting head 462 and further may communicate with the opening 409′ in a top surface in the cutting head 462.

In another embodiment, cutting head 465, shown in FIG. 104a, includes edges which provide three levels of cutting in mirror image relation, upper and lower proximal linear transverse cutting edges 466, 466′, upper and lower intermediate cutting edges 468, 468′, and upper and lower distal cutting edges 470, 470′, each set of edges forming a set of upper edges 475 and lower edges 475′. The members on which the edges 468 and 470 are formed each form a cooperating guide member for guiding the cutting edges symmetrically relative to the disc space. The three cutting edges are at different distances from the vertebrae and at different distances from the longitudinal axis. The cutting edges cut at different heights relating to the vertebrae and disc material and also cut at different points in the procedure of thrusting the chisel such that a first portion is cut, then the next portion is cut further away from the longitudinal axis of the instrument, and finally a third portion is cut furthest away from the longitudinal axis.

In FIGS. 105-109, a box chisel 500 with a scalpel 510 is shown. The chisel 500 includes a shank 502 having a handle 504 attached at a proximal end and a relatively enlarged cutting head 506 attached at a distal end. A passageway 508 extends contiguously along the axis 513 through the handle 504, shank 502, and cutting head 506.

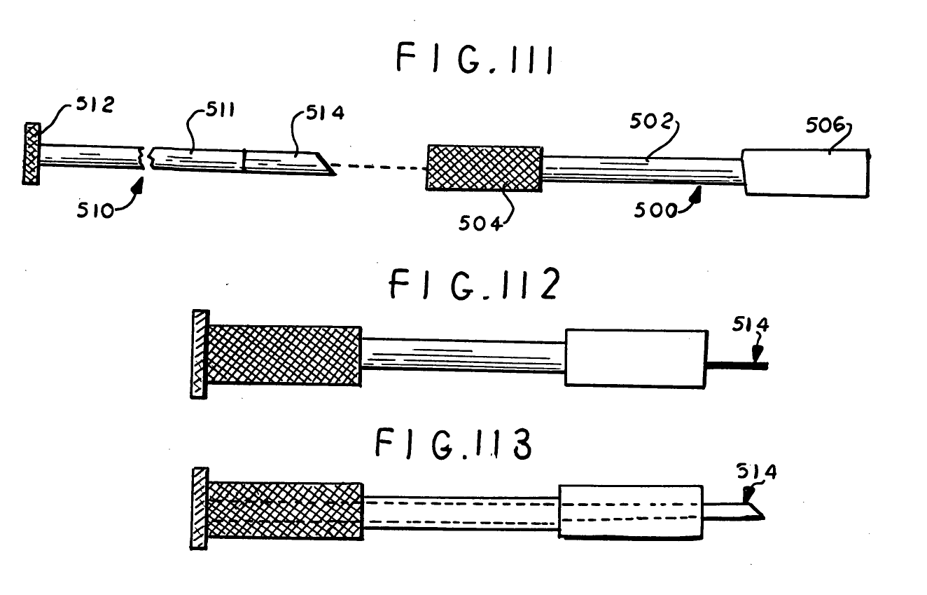

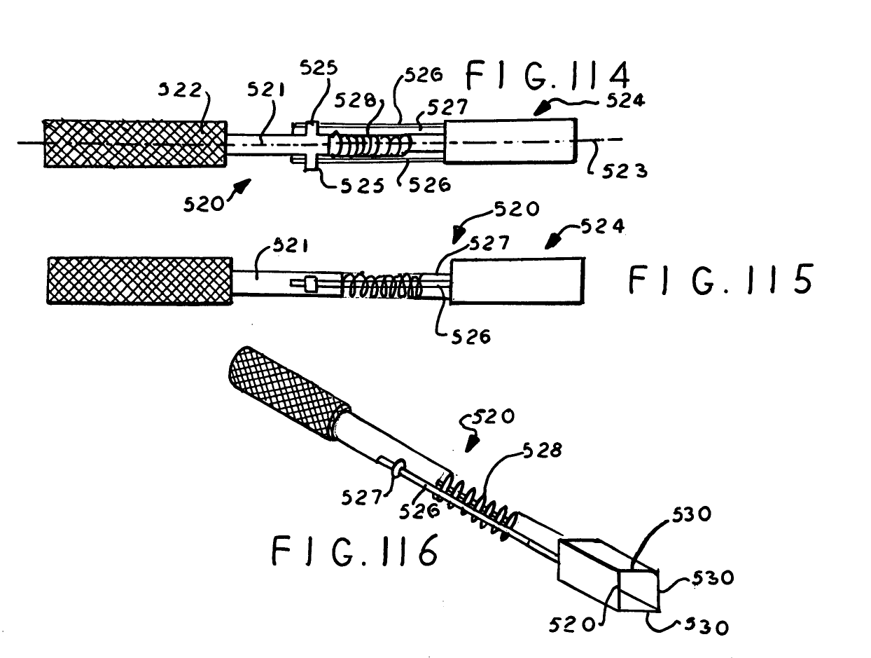

Scalpel 510, FIG. 108, has a blade 514, a shaft 511 and a knob disc shaped handle 512. The scalpel 510 passes into and through the passageway 508. The blade 514 of the scalpel 510 is attached to the side of the scalpel shaft 511 so that the blade 514 is off-center with respect to the longitudinal axis 501 of the shaft 511 and the chisel axis 513, FIGS. 109 and 110. The distal ends of the cutting head 506 form a quadrilateral box chisel terminating in cutting edges 516.

During surgery, the scalpel 510 is removably inserted into the box chisel 500 and used to cut material located between the cutting edges 516, as shown in FIGS. 111-113 by rotation of the knob handle 512. The scalpel 510 is then removed to provide enhanced manipulation of the box chisel 500 and/or to stop cutting in the area within the perimeter of the cutting edges 516.

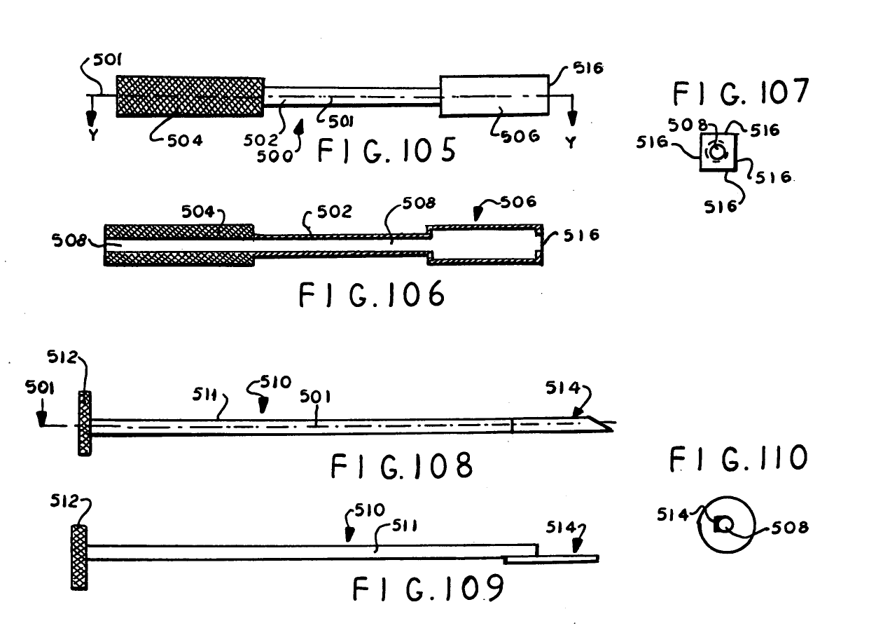

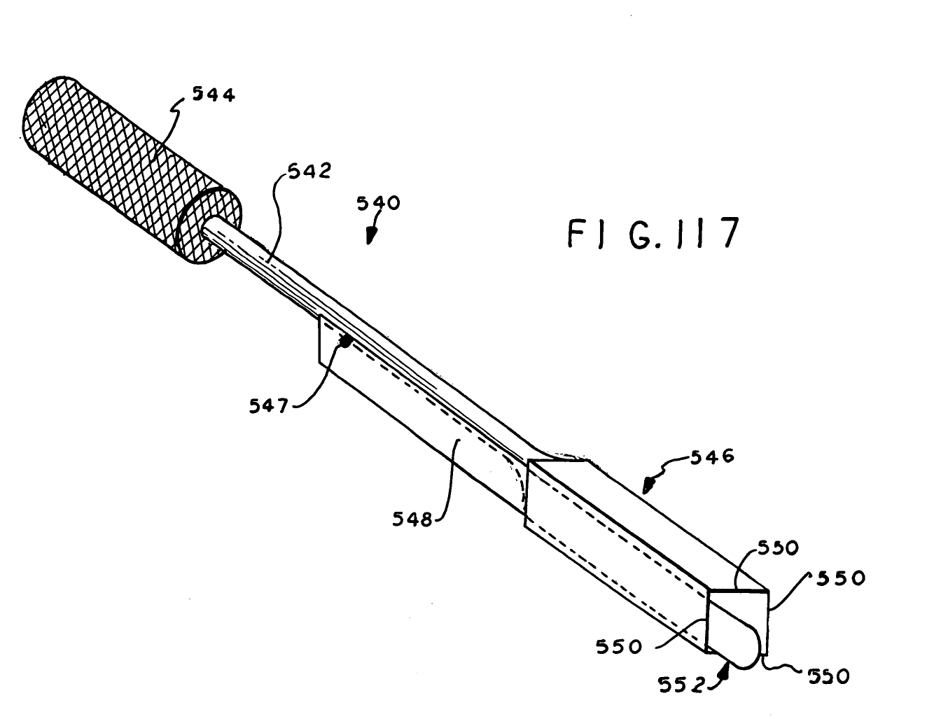

In FIGS. 114-116, a box chisel 520 including a spring 528 is shown. The box chisel includes a handle 522 and cutting head 524 as in the previous embodiment shown in FIGS. 105-106, however, the shaft 521 is foreshortened and spring 528 is positioned coextensive with and is affixed connected to the shaft 521 and also to the cutting head 524 at shaft-like extension member 527. Alternative resilient members may be used to perform similar function as the spring. The spring 528 is connected to the shaft 521 at one shaft end and to the chisel head between two elongated rods 526.

Rods 526 are slidably supported at one of their ends at two opposite sides of the shaft 521 to extension members 525 extending from the shaft 521. As a result the shaft 521 can displace longitudinally along the rods relative to the chisel head 524. The rods are affixed to the chisel head 520 at the opposite rod ends. The spring has a relatively high spring compression value to resiliently absorb shock from thrusting the chisel handle 522 or hammering the chisel handle so that a dampened thrust is received by the head and thus by the vertebrae via the cutting edges 530 at the distal end of the cutting head 524.

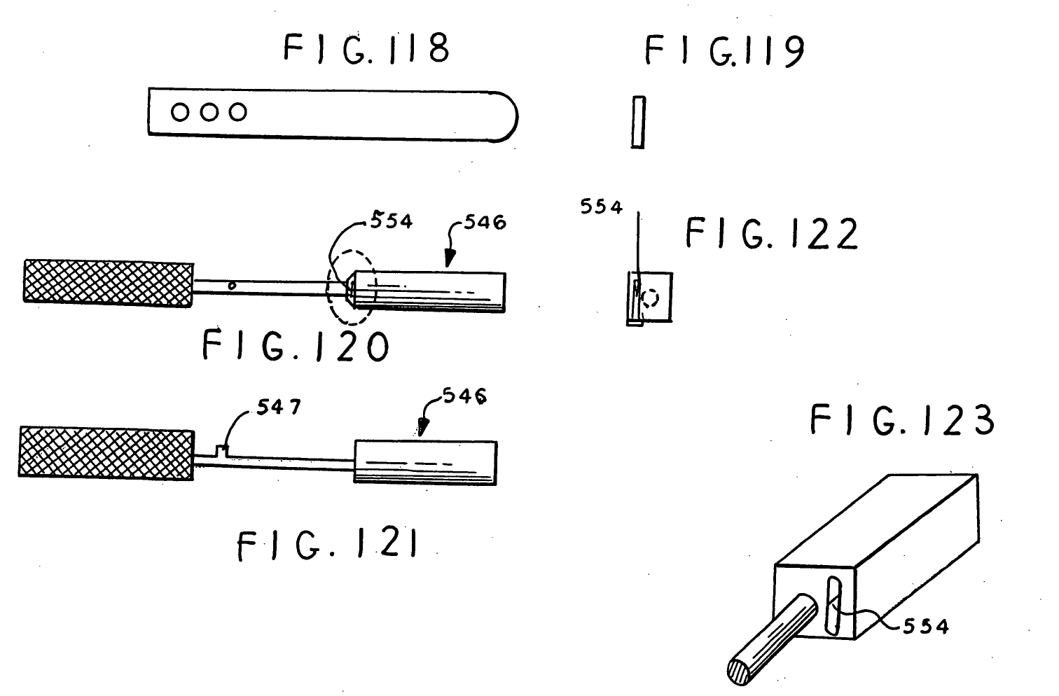

In FIGS. 117-123, a box chisel 540 is shown having a removable guide member 548, a shank 542 having a handle 544 attached at a proximal end, and a cutting head 546 attached at a distal end. The guide member 548 is planar sheet material such as metal or plastic, and is attached to a side of the shank 541 by an attachment stub 547. The guide member 548 passes through the cutting head 54 via an opening 554, FIG. 123, at the proximal end of the cutting head forming an extended guide portion 552. The guide portion 552 extends distally past the quadrilateral cutting edges 550 of the cutting head 546. The guide and thus portion 552 may be removably attached and selectively provided during surgery a guide function between adjacent vertebrae for guiding the cutting edges 550 of the box chisel 540 cutting head 546.

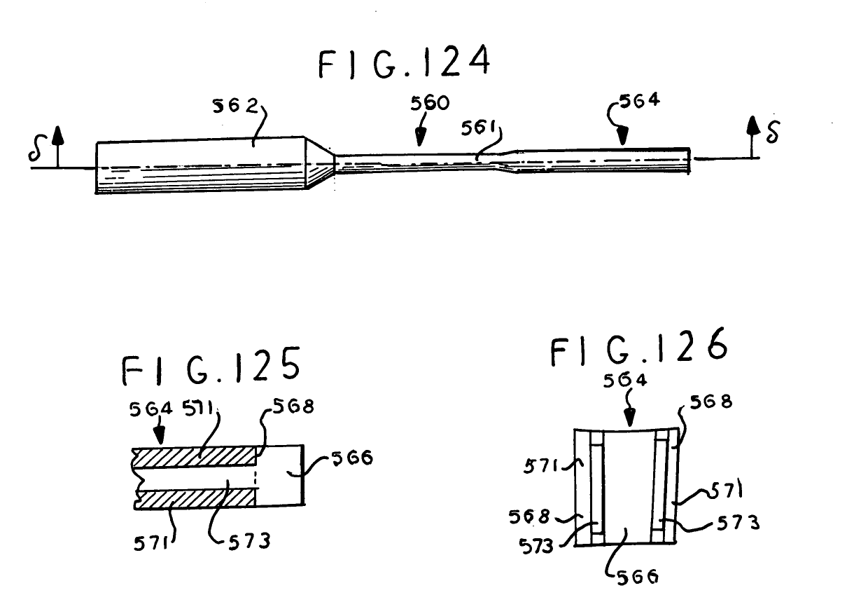

In another embodiment, an instrument 560 according to the present invention is shown in FIGS. 124-126. The shank 561 includes a handle 562 attached at a proximal end, and a cutting head 564 attached at a distal end as in previous embodiments. The cutting head 564 includes a guide member 566 extending distally and integral one piece with the body of the cutting head. The body of the cutting head includes side walls 571 distally terminating in transverse cutting edges 568 which are proximal in relation to the distal end of the guide member 566. A channel 573 is between walls 571.

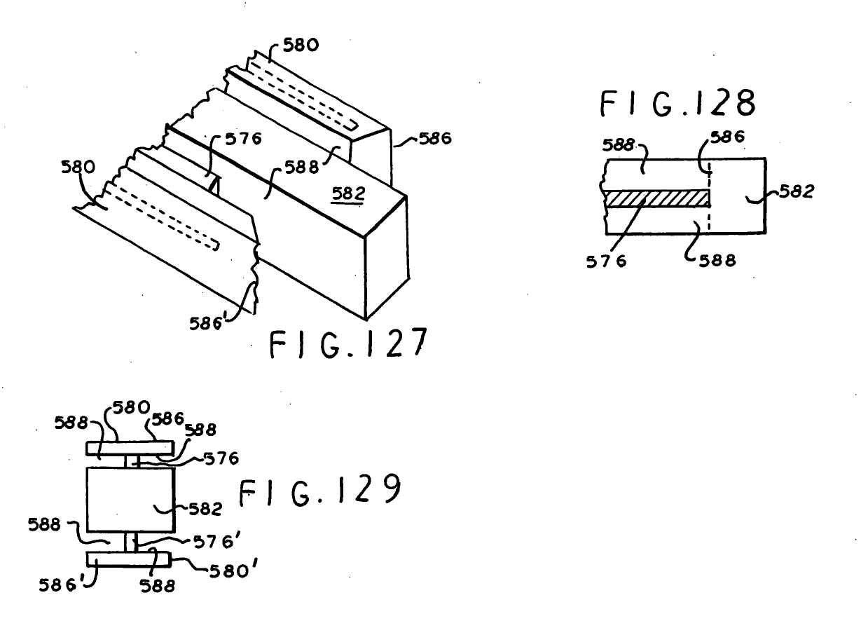

In FIGS. 127-129, in a further embodiment, planar side walls 580, 580′ extend externally the guide member 582 and terminate at cutting edges 586. The walls 580, 580′ are attached to the guide member and cutting head body 582 by strut members 576, 576′. The guide member 582 extends distally beyond the cutting edges 586 at the ends of the walls 580, 580′. The struts 576, 576′ form channels 588 between the side walls and the head and guide member 582.

It will occur to those of ordinary skill that various modifications may be made to the disclosed embodiments. It is intended that the invention be defined by the appended claims.

1. A chisel for preparing adjacent vertebrae for insertion of a spinal implant into the disc space defined by the vertebrae comprising:
a shank having a longitudinal axis extending in a first direction and distal and proximal ends;
a cutting head coupled to the distal end of the shank having a first wall terminating at a first cutting edge for cutting a channel in one of the vertebrae; and
a guide member extending from the head distal to the cutting edge for insertion into the disc space and having a first leg portion extending in the first direction and a second leg portion extending from the first portion in a direction transverse to the first direction and shaped for scooping cut material from between the vertebrae.

2. The chisel of claim 1 wherein the head has a side wall, the first portion of the guide member extending from the side wall, the member including a bend between the first and second portions.

3. The chisel of claim 1 wherein the cutting head includes a body defining an inner hollow portion, the body including a wall having an opening communicating with the inner hollow portion and communicating with a distal opening in the body to receive cut material.

4. The chisel of claim 1 wherein the cutting head includes a pair of opposing first cutting edges in spaced relation to the longitudinal axis, the guide member extending from between the first cutting edges and traverses the longitudinal axis.

5. The chisel of claim 1 wherein the guide member includes at least one second wall at a distal end terminating at a second cutting edge.

6. The chisel of claim 5 wherein the second wall of the guide member further includes at least one side cutting edge extending along a periphery of the guide member and contiguous with the second cutting edge.

7. The chisel of claim 5 wherein the second wall of the guide member is a second distance from the longitudinal axis which is less than the first distance from the longitudinal axis of the first cutting edge.

8. A chisel for preparing adjacent vertebrae for insertion of a spinal implant into the disc space defined by the vertebrae comprising:
a shank having a longitudinal axis extending in a first direction and distal and proximal ends; and
a cutting head coupled to the distal end of the shank, the cutting head including a body having at least one cantilevered first member extending in a direction substantially parallel to the first direction cantilevered a first distance from the longitudinal axis, the first cantilevered member distally terminating in a first cutting edge extending transverse to the first direction, the first cantilevered member and body defining an open region for providing visual access to a distal end of the cutting head from the head proximal end, the first cutting edge of the first cantilevered member for cutting one of the vertebra, the cutting head further including a guide member extending from the head distal the first cutting edge and positioned for bearing against adjacent vertebrae.

9. The chisel of claim 8 wherein the guide member includes at least one second wall at a distal end terminating with a second cutting edge.

10. The chisel of claim 8 wherein the second wall of the guide member terminates at a cutting edge extending along a periphery of the guide member contiguous with the second cutting edge.

11. The chisel of claim 10 wherein the second cutting edge is a second distance from the longitudinal axis which is less then the first distance.

12. The chisel of claim 8 wherein the first cutting edge of the at least one cantilevered first member includes a second cutting edge extending at an angle from the first cutting edge in a direction transverse to the direction of the longitudinal axis.

13. The chisel of claim 8 wherein the cutting head includes a pair of opposing cantilevered first members distally terminating in the first cutting edges in spaced relation about the longitudinal axis, and the first cutting edges each including second cutting edges extending substantially perpendicular to the first cutting edges toward one another and in a direction transverse to the direction of the longitudinal axis.

14. The chisel of claim 8 wherein the cutting head includes a pair of opposing first surfaces in spaced relation about the longitudinal axis, the guide member extending between the first surfaces along a plane defined by a side wall of the cutting head such that the opening extends between the first surfaces.

15. The chisel of claim 8 wherein the first cutting edge is convex.

16. The chisel of claim 8 wherein the first cutting edge is concave.

17. The chisel of claim 8 wherein the first cutting edge is curved in a direction that is transverse to the first direction.

18. The chisel of claim 12 wherein the pair of opposing first surfaces are curved toward one another in a direction transverse to the first direction.

19. A chisel for preparing adjacent vertebrae for insertion of a spinal implant into the disc space defined by the vertebrae, the chisel comprising:
a shank having a longitudinal axis extending in a first direction and distal and proximal ends; and
a cutting head coupled to the distal end of the shank, the cutting head including at least one first wall extending generally parallel to the longitudinal axis and being a first distance from the longitudinal axis, the first wall distally terminating with a first cutting edge lying in a plane for cutting a channel in one of the vertebra;
a guide member having at least one side wall and coupled distally to the head, the at least one side wall of the guide member extending substantially along a plane generally in the first direction, the guide member having a distal end wall transverse to the longitudinal axis, the guide member at least one side wall and distal end wall defining a substantially central opening therebetween.

20. The chisel of claim 19 wherein the guide member includes a peripheral surface around the substantially central opening, a second cutting edge extending along the periphery surface, the second cutting edge for removing material between adjacent vertebrae and extending distally beyond the first cutting edge.

21. The chisel of claim 19 wherein the opening in the guide member is a channel partially defined by a web portion extending between at least two opposite sides of the guide member, the cutting head includes an aperture communicating with the channel in the guide member.

22. The chisel of claim 19 wherein the first wall has an upper surface, the cutting head further including a lower second surface in spaced opposing relation to the upper surface with the longitudinal axis therebetween, the guide member central opening formed as a lower channel in opposing spaced relation to an upper channel, the cutting head further including a lower aperture in opposing spaced relation to an upper aperture and communicating with the respective upper and lower channels.

23. The chisel of claim 22 wherein the cutting head further includes upper and lower first walls in opposing spaced relation about the longitudinal axis, and each of the upper and lower first walls terminating in a first cutting edge in mirror image relationship with each other.

24. The chisel of claim 19 wherein the cutting edge of the first wall is V-shaped formed by two cutting edges interconnected at the apex of the V.

25. The chisel of claim 19 wherein the guide member defines a substantially central hollow opening extending therethrough and communicating with a substantially central opening in the head which extends through the head.

26. A chisel for preparing adjacent vertebrae for insertion of a spinal implant into the disc space defined by the vertebrae comprising:
a shank having a longitudinal axis extending in a first direction and distal and proximal ends;
a cutting head having proximal and distal ends with the proximal end coupled to the distal end of the shank, the cutting head including a first cutting portion comprising at least one first wall extending along a plane in the first direction and spaced a first distance to the longitudinal axis, the first wall terminating at a first cutting edge at the head distal end spaced from the axis and lying in a plane; and
a guide member extending from the head distal end in the first direction and having an end wall traversing the longitudinal axis, the guide member including a second cutting portion comprising a longitudinally extending second cutting edge extending distally beyond the first cutting edge, a second distance between the second cutting edge and the longitudinal axis being less than the first distance.

27. The chisel of claim 26 wherein the guide member further includes a plurality of side walls, the second cutting portion of the guide member including at least two second cutting edges in spaced relation at the side walls with the longitudinal axis therebetween.

28. The chisel of claim 26 wherein the second cutting portion of the guide member includes two upper cutting edges in spaced relation extending from a periphery of the side walls along substantially parallel planes with the longitudinal axis therebetween and two lower cutting edges in spaced relation extending from a periphery of the side wall in mirror image relation to the two upper cutting edges.

29. The chisel of claim 26 wherein the guide member has a hollow central region with opposing walls and includes a web portion extending between the opposing walls in the central region.

30. The chisel of claim 26 wherein the first cutting edge is serrated and lies in a direction that is transverse to the direction of the longitudinal axis.

31. The chisel of claim 26 wherein the guide member defines a substantially central opening and the head defines an opening having distal and proximal ends, the guide member opening communicating with the head opening at the opening distal end.

32. A chisel for preparing adjacent vertebrae for insertion of a spinal implant into the disc space defined by the vertebrae, the chisel comprising:
a shank having a longitudinal axis defining a longitudinal direction and distal and proximal ends; and
a cutting head coupled to the distal end of the shank, the cutting head having a body including a first cutting portion comprising two side walls extending in spaced relation to each other in the longitudinal direction and a top wall connecting the two side walls such that a first through opening is defined between the top wall and the side walls of the cutting head passing through the top and side walls in a longitudinal direction parallel to the longitudinal axis, the side walls and top wall terminating in first cutting edges distal to the shank for forming a channel in at least one of the vertebrae;
the cutting head further including a guide member one piece and integral with the body and positioned distal to the first cutting portion and extending substantially along the longitudinal axis, the guide member having a wall traversing the longitudinal axis, the guide member being coextensive with and an extension of a central portion of the head forming an interior surface of the first opening.

33. The chisel of claim 32 wherein the guide member is defined by a plurality of side walls, the guide member including a second cutting portion distal from the first cutting portion where the second cutting portion comprises at least one second cutting edge along the periphery of the wall of the guide member.

34. The chisel of claim 33 wherein the second cutting portion includes two upper cutting edges in spaced relation extending from the periphery of the side walls along substantially parallel planes with the longitudinal axis therebetween, the guide member including two lower cutting edges in spaced relation extending from a periphery of the side wall in mirror image relationship with the two upper cutting edges.

35. The chisel of claim 32 wherein the head side walls and a head bottom wall terminate in third cutting edges distally the shank for forming a channel in a second of said at least one of the vertebrae, the third cutting edges being a mirror image of the first cutting edges for forming a further opening which is a mirror image of the first opening.

36. The chisel of claim 32 wherein head has proximal and distal ends, the proximal end being coupled to the shank, the guide member extending from the head distal end and including a plurality of side walls, a top wall and a bottom wall defining a through chamber extending substantially along the longitudinal axis and communicating with a second opening in the cutting head adjacent to the shank at the head proximal end, the side, top and bottom walls of the guide member distally terminating in second cutting edges.

37. The chisel of claim 36 wherein the body of the cutting head has a top surface coextensive with a top surface of the guide member, the second opening being formed in the body top surface.

38. The chisel of claim 33 wherein the head has proximal and distal ends and top and bottom surfaces at the proximal end which is coupled to the shank, the guide member extending from the head distal end and including a plurality of side walls, a top wall and a bottom wall defining a through chamber extending substantially along the longitudinal axis and communicating with a second opening in the head top surface and with a third opening in the head bottom surface, the side, top and bottom walls of the guide member distally terminating in second cutting edges.

39. A chisel for preparing adjacent vertebrae for insertion of a spinal implant into the disc space defined by the vertebrae, the chisel comprising:
a shank having a longitudinal axis extending in a first direction and distal and proximal ends; and
a cutting head having distal and proximal ends, the proximal end being coupled to the distal end of the shank, the cutting head including a first cutting portion comprising at least one first wall extending in the first direction and spaced a first distance from the longitudinal axis, the first wall distally terminating at a first cutting edge at the head distal end transverse to the longitudinal axis and lying in a plane; and
a guide member extending in the first direction distally from the head distal end and having a second cutting portion positioned distal to the first cutting portion, the guide member second cutting portion having a distal end terminating at a second cutting edge extending transverse to the longitudinal axis.

40. The chisel of claim 39 wherein the at least one first wall includes multiple first cutting edges in stepped relation to one another.

41. The chisel of claim 39 wherein the cutting head has a top surface and includes a substantially central opening communicating with the distal end of the head and communicating with an opening in the top surface of the cutting head at the head proximal end.

42. The chisel of claim 39 wherein the first cutting portion at least one wall includes a first upper wall and a first lower wall in opposing spaced relation located a first distance from the longitudinal axis, the second cutting portion includes a second upper wall and a second lower wall in opposing spaced relation at a second distance from the longitudinal axis which is less than the first distance, the cutting head defining a substantially central opening which communicates with an opening in the first upper and lower walls at the proximal end of the cutting head and with at least one opening at the distal end of the cutting head.

43. The chisel of claim 39 wherein the head has a central opening, the first cutting portion defines a first opening extending through the first wall and communicating with the substantially central opening in the cutting head.

44. The chisel of claim 39 wherein the first cutting portion includes a second cutting edge having two legs converging at a distal apex.

45. The chisel of claim 39 wherein the second cutting portion includes a second cutting side edge extending along a periphery of the second cutting portion, the second cutting side edge having a portion lying in a plane perpendicular to the direction of the longitudinal axis.

46. The chisel of claim 39 wherein the second cutting portion includes upper and lower walls in spaced opposing relation to one another and including a side cutting edge extending contiguously with a periphery of the upper and lower walls.

47. The chisel of claim 39 wherein the second cutting portion includes upper and lower walls in spaced opposite relation to one another connected by a curved portion of the cutting head distal end to form a U-shaped peripheral cutting edge.

48. The chisel of claim 39 wherein the first cutting edge includes two legs converging at a distal apex.

49. The chisel of claim 39 wherein the cutting head has a roughened surface forming a rasp.

50. The chisel of claim 39 wherein the cutting head has a top surface having a plurality of raised sharpened projections.

51. The chisel of claim 39 wherein the cutting head includes a top surface having a plurality of raised cutting edges over a plurality of respective openings defined by the top surface such that a material shaved by the cutting edges falls through the openings.

52. The chisel of claim 39 wherein the second cutting portion of the guide member includes a surface having a plurality of raised cutting edges, the head having a central opening therethrough, the guide member having a plurality of respective openings that communicate with the central opening.

53. The chisel of claim 39 wherein the first cutting edge is curved toward the proximal end of the cutting head such that end portions of the cutting edge are positioned distally in relation to a center portion of the cutting edge.

54. The chisel of claim 39 wherein the first cutting edge is angled forming an apex such that the apex faces toward the proximal end of the cutting head.

55. The chisel of claim 39 wherein the second portion terminates in an arcuate cutting edge.

56. The chisel of claim 39 wherein the first portion wall terminates at a transverse linear cutting edge relative to the longitudinal axis and at an intermediate V-shaped cutting edge and the guide member second portion terminates at an arcuate or linear transverse cutting edge.

57. A chisel for preparing adjacent vertebrae for insertion of a spinal implant into the disc space defined by the vertebrae, the chisel comprising:
a shank having a longitudinal axis and distal and proximal ends;
a handle portion coupled to the proximal end of the shank;
a cutting head coupled to the distal end of the shank, the cutting head including a hollow body contiguous with an opening through the shank and an opening through the handle, the body of the cutting head comprising four sides distally terminating in cutting edges;
a scalpel comprising a knob at a proximal end and a blade edge at a distal end, the scalpel being removable positioned through the contiguous openings of the handle portion, the shank, and the hollow body of the cutting head such that the blade edge extends distal to the cutting edges of the cutting head.

58. The chisel of claim 57 wherein the scalpel comprises a blade portion distally terminating in the blade edge, the blade portion coupled to a side of the distal end of the scalpel and extending along a plane substantially parallel to a longitudinal axis of the scalpel.

59. The chisel of claim 57 wherein the blade portion is planar and defines a width extending normal to the plane of the blade portion.

60. The chisel of claim 57 wherein the blade portion is planar and defines a width extending horizontally.

61. A chisel for preparing adjacent vertebrae for insertion of a spinal implant into the disc space defined by the vertebrae, the chisel comprising:
a shank having a longitudinal axis and distal and proximal ends;
a handle coupled to the proximal end of the shank;
a cutting head coupled to the distal end of the shank, the cutting head including a hollow body comprising four sides each distally terminating in a cutting edge; and
a resilient member positioned between and for resiliently coupling a first proximal portion of the shank to a second distal portion of the shank.

62. The chisel of claim 61 wherein the resilient member is a spring.

63. The chisel of claim 61 further comprising at least one supporting rod extending in a plane substantially parallel to the longitudinal axis and slidably connected to the shank, the supporting rod being fixedly connected to a proximal end of the cutting head.

64. The chisel of claim 61 further comprising an elongated guide extending substantially parallel to the longitudinal axis and fixedly attached to the shank, a distal portion of the elongated guide element extending through the hollow body of the cutting head and distally terminating past the cutting edges and forming a planar element for insertion between adjacent vertebrae.

65. The chisel of claim 61 wherein the elongated guide element passes through a proximate opening in the hollow body of the cutting head.

66. The chisel of claim 61 wherein the elongated guide element is planar and defines a width extending horizontally.

67. The chisel of claim 61 wherein the elongated guide element is planar and defines a width extending vertically.

68. A chisel for preparing adjacent vertebrae for insertion of a spinal implant into the disc space defined by the vertebrae, the chisel comprising:
a shank having a longitudinal axis and distal and proximal ends; and
a cutting head coupled to the distal end of the shank, the cutting head including upper and lower spaced walls each defining a first cutting portion distally terminating with a first cutting edge, the first cutting edges being located with the longitudinal axis therebetween; and
a guide member extending distal to the first cutting portion and extending coextensive with the shank between the upper and lower spaced walls, the guide member further including top and bottom surfaces in spaced relation with each other and at a second distance from the longitudinal axis which is less than a first distance between the first cutting edges and the longitudinal axis, the cutting head further including upper and lower openings communicating with each other and extending through the cutting head, the upper and lower openings being defined by the upper and lower walls of the first cutting portions and by the top and bottom surfaces of the guide member.

69. The chisel of claim 68 wherein a plurality of support structures extend between the upper and lower walls and the guide member along a periphery of the cutting head and further defining the upper and lower openings.

70. The chisel of claim 68 wherein an upper support structure extends between the upper wall and the guide member away from a periphery of the cutting head such that a plurality of upper openings are defined by the upper support structure and the periphery of the cutting head, and a lower support structure extends between the lower wall and the guide member away from the periphery of the cutting head such that a plurality of lower openings are defined by the lower support structure and the periphery of the cutting head.

71. A method for preparation of a disc space for insertion of a spinal implant into the disc space between adjacent vertebrae, comprising the steps of:
positioning an extended guide member between adjacent vertebrae, the guide member being integral with a cutting head connected to a shank of a chisel instrument, the guide member directing first cutting edges of the cutting head into position with desired vertebrae;
removing a first portion of endplate material from at least one of two adjacent vertebrae using the first cutting edges of the cutting head; and
removing a second portion of disc material between the adjacent vertebrae using at least one second cutting edge on a distal end of the guide member.

72. The method of claim 71 further including the step of:
scraping material from adjacent vertebrae using a third cutting edge extending along the periphery of the guide member by twisting the chisel about a longitudinal axis to displace the guide member in an arcuate path.

73. The method of claim 71 wherein the step of removing the second portion of material further includes the second portion of material being at a less distance to a longitudinal axis of the shank than the first portion of material such that the first cutting edges are positioned a first distance from the longitudinal axis to remove the first portion of material and the second cutting edges are positioned a second distance from the longitudinal axis which is less than the first distance and positions the at least one second cutting edge to remove the second portion of material.

74. A vertebral bone cutting chisel comprising:
a bone cutting head having proximal and distal ends and opposing first and second upper and lower walls, the proximal end for coupling to an instrument shank extending in a first direction, the distal end of said walls terminating in first edges extending transverse to the first direction; and
a pair of spaced guide members extending from said head at and distally said first edges and terminating at second transverse edges, the guide members defining an open region therebetween, the first and second edges forming a stepped relation with each other.

75. The chisel of claim 74 wherein the guide members are contiguous with the first edges.

76. The chisel of claim 74 wherein the head has a through chamber, the chamber having a distal opening in communication with the open region.

77. The chisel of claim 76 wherein the guide members each have an opening therethrough in communication with the open region.

78. The chisel of claim 74 further including an intermediate third edge disposed between the first and second edges in mirror image relationship to each other.

79. The chisel of claim 78 wherein the intermediate edge is linear and transverse to the first direction.

80. The chisel of claim 78 wherein the intermediate edge is V-shaped.

81. The chisel of claim 74 wherein the first edges are concave.

82. The chisel of claim 74 wherein the first edges are convex.

83. A vertebral bone cutting chisel comprising:
a bone cutting head having proximal and distal ends and opposing first and second upper and lower walls connected to and cantilevered from a side wall, the proximal end for coupling to an instrument shank extending in a first direction, the distal end of said walls terminating in cantilevered first and second edges extending transverse to the first direction; and
a guide member extending from said head side wall distally said first edge.

84. The chisel of claim 83 wherein the side walls are arcuate and in mirror image relation extending about a common axis.

85. The chisel of claim 83 wherein the first and second edges are each one of concave or convex.
